(19) United States
(12) Patent Application Publication
(10) Pub. No.: US 2018/0250061 A1
(43) Pub. Date: Sep. 6, 2018

(54) THERMAL ENERGY TREATMENT APPARATUS AND METHOD OF OPERATING THERMAL ENERGY TREATMENT APPARATUS

(71) Applicant: OLYMPUS CORPORATION, Tokyo (JP)

(72) Inventor: Hiroki KAZUNO, Tokyo (JP)

(73) Assignee: OLYMPUS CORPORATION, Tokyo (JP)

(21) Appl. No.: 15/974,107

(22) Filed: May 8, 2018

Related U.S. Application Data
(63) Continuation of application No. PCT/JP2015/084191, filed on Dec. 4, 2015.

(51) Int. Cl. A61B 18/08

(52) U.S. Cl. CPC A61B 18/082; A61B 2018/00702; A61B 18/085

(57) ABSTRACT

A thermal energy treatment apparatus includes: an insulating substrate having a longitudinal axis; a heating element provided on the insulating substrate, the heating element including a heater configured to generate heat by energization, a resistance value of the heater having per unit length in a direction of the longitudinal axis being a first resistance value, and a connector configured to be conducted to the heater, a resistance value of the connector per unit length in the direction of the longitudinal axis being a second resistance value smaller than the first resistance value; and a controller configured to determine a state of the connector based on an index value of a temperature of the connector, and restrict an output value of energization of the heater based on the determined state of the connecting unit.

### CROSS-REFERENCE TO RELATED APPLICATION

This application is a continuation of International Application No. PCT/JP2015/084191, filed on Dec. 4, 2015, the entire contents of which are incorporated herein by reference.

### BACKGROUND

The present disclosure relates to a thermal energy treatment apparatus and a method of operating a thermal energy treatment apparatus.

In the related art, there is known a thermal energy treatment apparatus (thermal tissue surgery system) for treatment (joining (or anastomosis), dissection, or the like) of a living tissue by applying energy to the living tissue (refer to JP 2012-24583 A, for example).

The thermal energy treatment apparatus described in JP 2012-24583 A includes a pair of jaws to grasp a living tissue. The pair of jaws includes a therapeutic energy applying structure to generate thermal energy.

For example, in order to reduce the thickness of such a therapeutic energy applying structure, it is conceivable to adopt a configuration using a flexible substrate and a heat transfer plate described below.

The flexible substrate functions as a sheet heater. One surface of the flexible substrate includes a heating unit that generates heat by energization and a connecting unit that conducts to the heating unit.

The heat transfer plate is formed of a conductor such as copper. The heat transfer plate is arranged to face one surface (heating unit) of the flexible substrate, and transmits heat from the heating unit to the living tissue (applies thermal energy to the living tissue).

Note that the flexible substrate is longer than the heat transfer plate, and when assembled, one end side (side where the connecting unit is provided) protrudes from the heat transfer plate. In addition, a lead wire for supplying power to the heating unit is connected to the connecting unit provided on the one end side. That is, locating the lead wire on one side of the flexible substrate (side on which the heat transfer plate is arranged) makes it possible to reduce the thickness of the therapeutic energy applying structure.

### SUMMARY

A thermal energy treatment apparatus according to one aspect of the present disclosure includes: an insulating substrate having a longitudinal axis; a heating element provided on the insulating substrate, the heating element including a heater configured to generate heat by energization, a resistance value of the heater having per unit length in a direction of the longitudinal axis being a first resistance value, and a connector configured to be conducted to the heater, a resistance value of the connector per unit length in the direction of the longitudinal axis being a second resistance value smaller than the first resistance value; and a controller configured to determine a state of the connector based on an index value of a temperature of the connector, and restrict an output value of energization of the heater based on the determined state of the connecting unit.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating a thermal energy treatment apparatus according to a first embodiment;

FIG. 2 is an enlarged view of a distal end portion of an energy treatment tool illustrated in FIG. 1;

FIG. 3 is a diagram illustrating a therapeutic energy applying structure illustrated in FIG. 2;

FIG. 4 is a diagram illustrating the therapeutic energy applying structure illustrated in FIG. 2;

FIG. 5 is a block diagram illustrating a configuration of a control apparatus illustrated in FIG. 1;

FIG. 6 is a flowchart illustrating operation of the control apparatus illustrated in FIG. 5;

FIG. 7 is a diagram illustrating an example of a waveform of a power value output to a heating element by the operation of the control apparatus illustrated in FIG. 5;

FIG. 8 is a block diagram illustrating a configuration of a control apparatus constituting a thermal energy treatment apparatus according to modification 1-1 of the first embodiment;

FIG. 9 is a flowchart illustrating operation of the control apparatus illustrated in FIG. 8;

FIG. 10 is a diagram illustrating an example of a waveform of a power value output to a heating element by the operation of the control apparatus illustrated in FIG. 9;

FIG. 11 is a block diagram illustrating a configuration of a control apparatus constituting a thermal energy treatment apparatus according to modification 1-2 of the first embodiment;

FIG. 12 is a flowchart illustrating operation of the control apparatus illustrated in FIG. 11;

FIG. 13 is a diagram illustrating an example of a waveform of a power value output to a heating element by the operation of the control apparatus illustrated in FIG. 12;

FIG. 14 is a block diagram illustrating a configuration of a control apparatus constituting a thermal energy treatment apparatus according to modification 1-3 of the first embodiment;

FIG. 15 is a block diagram illustrating a configuration of a control apparatus constituting a thermal energy treatment apparatus according to a second embodiment;

FIG. 16 is a flowchart illustrating operation of the control apparatus illustrated in FIG. 15;

FIG. 17 is a block diagram illustrating a configuration of a control apparatus constituting a thermal energy treatment apparatus according to modification 2-1 of the second embodiment;

FIG. 18 is a flowchart illustrating operation of the control apparatus illustrated in FIG. 17;

FIG. 19 is a block diagram illustrating a configuration of a control apparatus constituting a thermal energy treatment apparatus according to modification 2-2 of the second embodiment;

FIG. 20 is a flowchart illustrating operation of the control apparatus illustrated in FIG. 19;

FIG. 21 is a block diagram illustrating a configuration of a control apparatus constituting a thermal energy treatment apparatus according to a third embodiment;

FIG. 22 is a diagram illustrating the therapeutic energy applying structure illustrated in FIG. 21;

FIG. 23 is a flowchart illustrating operation of the control apparatus illustrated in FIG. 21;

FIG. 24A is a diagram illustrating step S6G illustrated in FIG. 23;

FIG. 24B is a diagram illustrating step S6G illustrated in FIG. 23;

FIG. 25 is a block diagram illustrating a configuration of a control apparatus constituting a thermal energy treatment apparatus according to modification 3-1 of the third embodiment;

FIG. 26 is a flowchart illustrating operation of the control apparatus illustrated in FIG. 25;

FIG. 27 is a flowchart illustrating power limit flag determination processing (step S18) illustrated in FIG. 26; and

FIG. 28 is a flowchart illustrating impedance flag determination processing (step S19) illustrated in FIG. 26.

### DETAILED DESCRIPTION

Hereinafter, embodiments will be described with reference to the drawings. Note that the present disclosure is not limited by the following embodiments. In the drawings, same reference signs are attached to the same portions.

### First Embodiment

Schematic configuration of thermal energy treatment apparatus

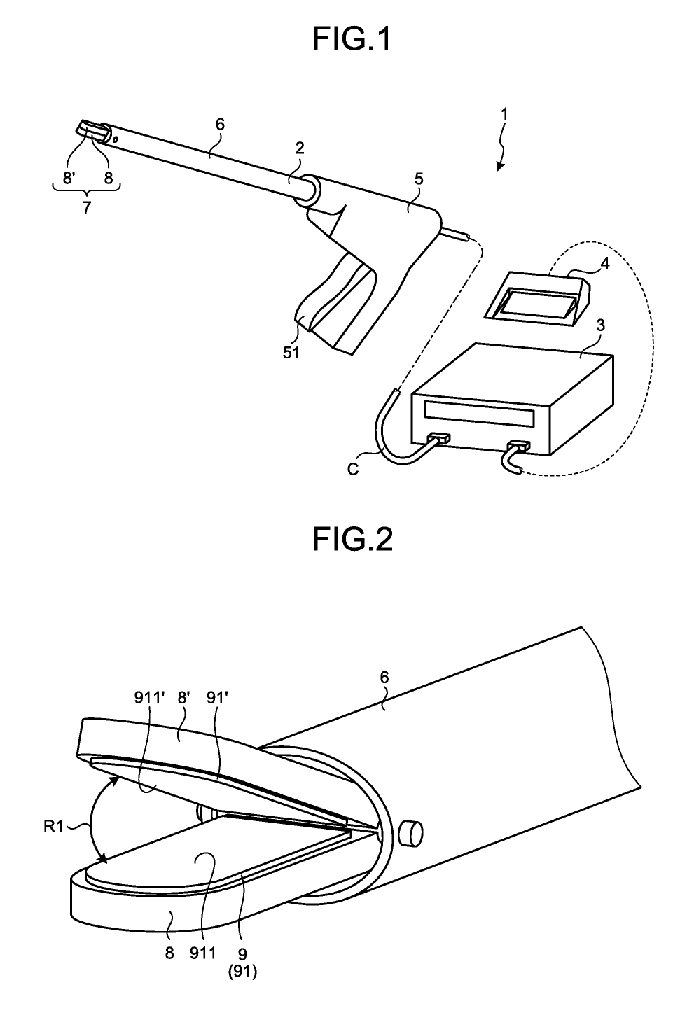

FIG. 1 is a diagram schematically illustrating a thermal energy treatment apparatus 1 according to a first embodiment.

The thermal energy treatment apparatus 1 applies energy to a living tissue as a treatment target to provide treatment (joining (or anastomosis) and dissection) to the living tissue. As illustrated in FIG. 1, the thermal energy treatment apparatus 1 includes an energy treatment tool 2, a control apparatus 3, and a foot switch 4.

Configuration of Energy Treatment Tool

The energy treatment tool 2 is a linear-type surgical medical treatment tool for performing treatment on a living tissue through an abdominal wall, for example. As illustrated in FIG. 1, the energy treatment tool 2 includes a handle 5, a shaft 6, and a grasping unit 7.

The handle 5 is a portion to be gripped by an operator. As illustrated in FIG. 1, the handle 5 includes an operation knob 51.

As illustrated in FIG. 1, the shaft 6 has a substantially cylindrical shape, and one end thereof is connected to the handle 5. The grasping unit 7 is attached to the other end of the shaft 6. The shaft 6 internally includes an open-close mechanism (not illustrated) for opening and closing the holding members 8 and 8′ (FIG. 1) constituting the grasping unit 7 in accordance with operation of the operation knob 51 by the operator. Moreover, the shaft 6 internally also includes an electric cable C (FIG. 1) connected to the control apparatus 3, being arranged from one end side to the other end side via the handle 5.

Configuration of Grasping Unit

FIG. 2 is an enlarged view of a distal end portion of the energy treatment tool 2.

In FIG. 1 and FIG. 2, the configuration indicated by the sign without “′” is the same as the configuration indicated by the sign with “′”.

The grasping unit 7 is a portion that grasps a living tissue for treatment of the living tissue. As illustrated in FIG. 1 or FIG. 2, the grasping unit 7 includes a pair of holding members 8 and 8′.

The pair of holding members 8 and 8′ is pivotably supported on the other end of the shaft 6 so as to be opened and closed in a direction of an arrow R1 (FIG. 2), enabling grasping of the living tissue in accordance with the operation of the operation knob 51 by the operator.

Among the pair of holding members 8 and 8′, the holding member 8 arranged on the lower side includes a therapeutic energy applying structure 9 as illustrated in FIG. 2. Moreover, on a lower surface of the holding member 8′ arranged on the upper side, a heat transfer plate 91′ (including a treatment surface 911′) similar to a heat transfer plate 91 (including a treatment surface 911) to be described below, constituting the therapeutic energy applying structure 9′ is attached.

Configuration of Therapeutic Energy Applying Structure

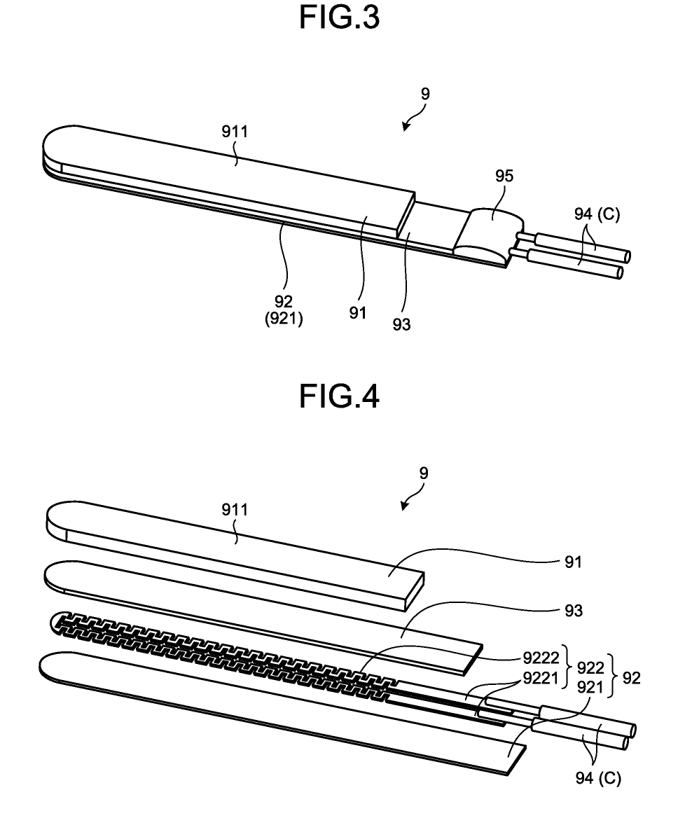

FIGS. 3 and 4 are diagrams illustrating the therapeutic energy applying structure 9. Specifically, FIG. 3 is a perspective view of the therapeutic energy applying structure 9 viewed from the upper side in FIG. 2. FIG. 4 is an exploded perspective view of FIG. 3.

The therapeutic energy applying structure 9 is attached to the upper side surface of the holding member 8 in FIGS. 1 and 2. The therapeutic energy applying structure 9 applies thermal energy to a living tissue under the control of the control apparatus 3. As illustrated in FIG. 3 or FIG. 4, the therapeutic energy applying structure 9 includes the heat transfer plate 91, a flexible substrate 92, an adhesive sheet 93, and two lead wires 94.

The heat transfer plate 91 is an elongated thin plate (elongated shape extending in the left-right direction (longitudinal axis direction) in FIGS. 3 and 4) formed of a material such as copper. In a state where the therapeutic energy applying structure 9 is attached to the holding member 8, the treatment surface 911 as one plate surface faces the holding member 8′ side (the upper side in FIGS. 1 and 2). In a state where the living tissue is grasped by the holding members 8 and 8′, the heat transfer plate 91 allows the treatment surface 911 to come into contact with the living tissue to transmit the heat from the flexible substrate 92 to the living tissue (apply thermal energy to the living tissue).

A portion of the flexible substrate 92 generates heat, and the flexible substrate 92 functions as a sheet heater to heat the heat transfer plate 91 by the generated heat. As illustrated in FIG. 3 or FIG. 4, the flexible substrate 92 includes an insulating substrate 921 and a heating element 922 (FIG. 4).

The insulating substrate 921 is an elongated sheet (having elongated shape extending in the left-right direction (longitudinal axis direction) in FIGS. 3 and 4) formed of polyimide as an insulating material.

Note that the material of the insulating substrate 921 is not limited to polyimide, and a high heat-resistant insulating material such as aluminum nitride, alumina, glass, and zirconia may be employed, for example.

The width dimension of the insulating substrate 921 is set to be substantially the same as the width dimension of the heat transfer plate 91. Moreover, the length dimension of the insulating substrate 921 (length dimension in the longitudinal axis direction in FIGS. 3 and 4) is set to be longer than the length dimension (length dimension in the longitudinal axis direction in FIG. 4) of the heat transfer plate 91.

The heating element 922 is formed of stainless steel (SUS 304) being a conductive material, and includes a pair of lead wire connecting units (connectors) 9221 and a heating unit (heater) 9222 as illustrated in FIG. 4. The heating element 922 is bonded to one surface of the insulating substrate 921 by thermal compression bonding.

Note that the material of the heating element 922 is not limited to the stainless steel (SUS 304), and may be another stainless material (for example, 400 series), or a conductive material such as platinum or tungsten. Moreover, the heating element 922 is not limited to a configuration of being bonded to one surface of the insulating substrate 921 by thermal compression bonding, and may employ a configuration of being formed on the one surface by vapor deposition or the like.

The pair of lead wire connecting units 9221 has a function as a connector according to the present disclosure, and extends from one end side (right end side in FIG. 4) of the insulating substrate 921 to the other end side (left end side in FIG. 4), being provided so as to face each other along a width direction of the insulating substrate 921. Each of the two lead wires 94 (FIGS. 3 and 4) constituting the electric cable C is joined (connected) to each of the pair of lead wire connecting units 9221.

The heating unit 9222 has one end connected (conducted) to one of the lead wire connecting units 9221, and meanders in a wave shape with a fixed line width from the one end, and extends along a U shape following an outer edge shape of the insulating substrate 921, with the other end being connected (conducted) to the other of the lead wire connecting units 9221.

A voltage is applied (energized) to the pair of lead wire connecting units 9221 by the control apparatus 3 via the two lead wires 94, leading to generation of heat in the heating unit 9222.

Note that in the first embodiment, the pair of lead wire connecting units 9221 has an electric resistance value (second resistance value) per unit length in the longitudinal axis direction smaller than the electric resistance value (first resistance value) of the heating unit 9222 per unit length in the longitudinal axis direction.

As illustrated in FIG. 3 or FIG. 4, the adhesive sheet 93 is interposed between the heat transfer plate 91 and the flexible substrate 92, and adhesively secures a surface on the heat transfer plate 91 on the side opposite to the treatment surface 911 with one surface (surface on the heating element 922 side) of the flexible substrate 92 in a state where a portion of the flexible substrate 92 protrudes from the heat transfer plate 91. The adhesive sheet 93 is a sheet having excellent heat conductivity, insulating property, high heat resistance, and adhesive property with an elongated shape (extending in the left-right direction (longitudinal axis direction) in FIGS. 3 and 4), and is formed by mixing a high thermal conductive filler (non-conductive material) such as alumina, boron nitride, graphite, or aluminum nitride with a resin such as epoxy or polyurethane.

Note that the width dimension of the adhesive sheet 93 is set to be substantially the same as the width dimension of the insulating substrate 921. Moreover, the length dimension of the adhesive sheet 93 (length dimension in the longitudinal axis direction in FIGS. 3 and 4) is set to be longer than the length dimension of the heat transfer plate 91 (length dimension in the longitudinal axis direction in FIGS. 3 and 4) and set to be shorter than the length dimension of the insulating substrate 921 (length dimension in the longitudinal axis direction in FIGS. 3 and 4).

Positional Relationship Between Heat Transfer Plate, Flexible Substrate, and Adhesive Sheet

Next, the positional relationship between the heat transfer plate 91, the flexible substrate 92, and the adhesive sheet 93 will be described.

The heat transfer plate 91 is arranged so as to cover the entire region of the heating unit 9222 and to expose the pair of lead wire connecting units 9221.

The adhesive sheet 93 is arranged so as to cover the entire region of the heating unit 9222 and a portion of the pair of lead wire connecting units 9221. That is, one end side in the longitudinal direction (right end side in FIGS. 3 and 4) of the adhesive sheet 93 projects to the right side in FIGS. 3 and 4 with respect to the heat transfer plate 91. The two lead wires 94 are joined (connected) to an externally exposed region on the pair of lead wire connecting units 9221 (region not covered with the adhesive sheet 93).

Moreover, after the two lead wires 94 are individually joined (connected), the externally exposed region on the pair of lead wire connecting units 9221 (region not covered with the adhesive sheet 93) undergoes application of an insulating member 95 (FIG. 3), so as to be sealed together with the two lead wires 94. Accordingly, the heating element 922 is insulated and sealed on the insulating substrate 921 by the adhesive sheet 93 and the insulating member 95.

Configuration of Control Apparatus and Foot Switch

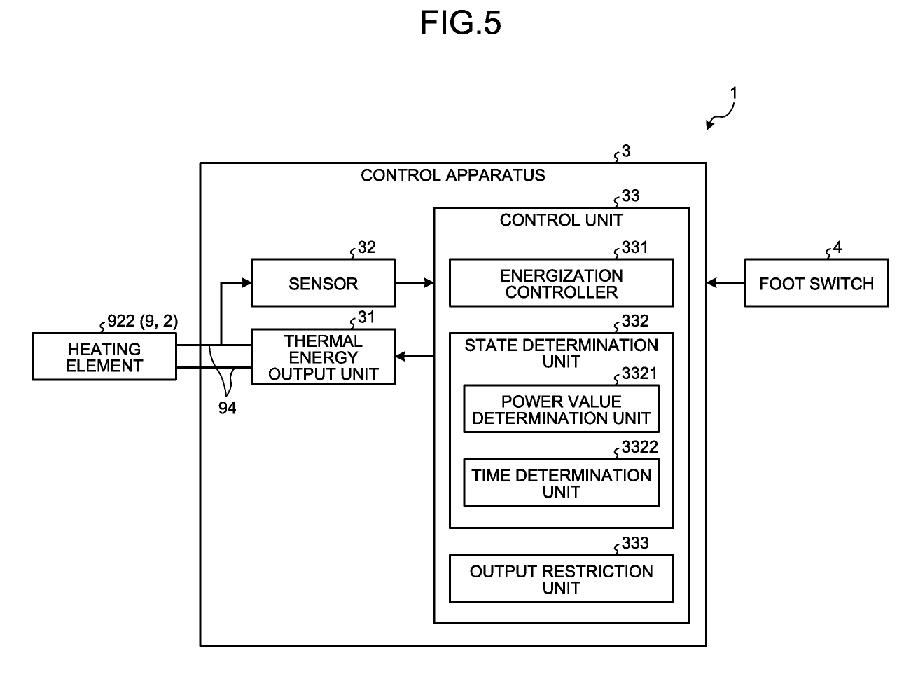

FIG. 5 is a block diagram illustrating a configuration of the control apparatus 3.

Note that FIG. 5 mainly illustrates major portions as the configuration of the control apparatus 3.

The foot switch 4 has a function as an operation receiver according to the present disclosure and receives first user operation of shifting the energy treatment tool 2 from a standby state (state in which energization to the heating element 922 is stopped) to an energized state (state in which the heating element 922 is energized), and receives second user operation of shifting the energy treatment tool 2 from the energized state to the standby state. In the first embodiment, the foot switch 4 receives the first user operation by being pressed down by an operator's foot (switch on), and receives the second user operation by release of the operator's foot from the foot switch 4 (switch off). The foot switch 4 subsequently outputs a signal corresponding to the first and second user operations to the control apparatus 3.

Note that the operation receiving unit according to the present disclosure is not limited to the foot switch 4, and other manual operation switches or the like may be employed.

The control apparatus 3 totally controls the operation of the energy treatment tool 2. As illustrated in FIG. 5, the control apparatus 3 includes a thermal energy output unit 31, a sensor 32, and a control unit (controller) 33.

Under the control of the control unit 33, the thermal energy output unit 31 applies a voltage (energizes) to the heating element 922 via the two lead wires 94. The thermal energy output unit 31 has a function as an output unit according to the present disclosure.

The sensor 32 detects a current value and a voltage value supplied (energized) from the thermal energy output unit 31 to the heating element 922. The sensor 32 subsequently outputs a signal corresponding to the detected current value and voltage value to the control unit 33. Note that the sensor 32 according to the first embodiment has a function as a first detection unit according to the present disclosure.

The control unit 33 includes a central processing unit (CPU). When the foot switch 4 is turned on, the control unit 33 executes feedback control of the heating element 922 in accordance with a predetermined control program. As illustrated in FIG. 5, the control unit 33 includes an energization controller 331, a state determination unit 332, and an output restriction unit 333.

When the foot switch 4 is turned on, the energization controller 331 activates the thermal energy output unit 31 to start energizing the heating element 922 so as to switch the energy treatment tool 2 to the energized state. In the first embodiment, the energization controller 331 (thermal energy output unit 31) is configured to energize the heating element 922 with direct current in the energized state. Then, while grasping the temperature of the heat transfer plate 91 in the energized state, the energization controller 331 performs feedback control of the heating element 922 (control of output value (power value) to be supplied (energized) to the heating element 922) so as to set the heat transfer plate 91 to a target temperature. Moreover, when the foot switch 4 is turned off, the energization controller 331 stops operation of the thermal energy output unit 31 to stop energization to the heating element 922 so as to switch the energy treatment tool 2 to the standby state.

As the temperature of the heat transfer plate 91 used in the feedback control, for example, the following temperatures may be employed.

For example, a resistance value of the heating element 922 is obtained based on the current value and voltage value (current value and voltage value detected by the sensor 32) supplied (energized) from the thermal energy output unit 31 to the heating element 922. Then, the resistance value of the heating element 922 is converted into temperature, and the converted temperature is used as the temperature of the heat transfer plate 91.

Moreover, for example, a temperature sensor including a thermocouple, a thermistor or the like is provided on the heat transfer plate 91 or the like, and the temperature detected by the temperature sensor is used as the temperature of the heat transfer plate 91.

The state determination unit 332 determines the state of the pair of lead wire connecting units 9221 based on an index value serving as an index of the temperature of the pair of lead wire connecting units 9221. In the first embodiment, the index value is a power value supplied (energized) to the heating element 922. As illustrated in FIG. 5, the state determination unit 332 includes a power value determination unit 3321 and a time determination unit 3322.

The power value determination unit 3321 calculates the power value supplied (energized) to the heating element 922 based on the current value and the voltage value detected by the sensor 32. Subsequently, the power value determination unit 3321 compares the calculated power value with a preset steady state power limit value (equivalent to a first threshold according to the present disclosure), and clocks the time during which the power value continuously exceeds the steady state power limit value (hereinafter referred to as “clocked time”). That is, the power value determination unit 3321 has a function as an index value determination unit according to the present disclosure.

The time determination unit 3322 compares the clocked time with a preset continuation time limit (equivalent to a second threshold according to the present disclosure), and determines whether the clocked time has exceeded the continuation time limit.

When the time determination unit 3322 determines that the clocked time has exceeded the continuation time limit, the output restriction unit 333 controls operation of the thermal energy output unit 31 to restrict the output value (power value) to be supplied (energized) to the heating element 922.

Operation of Control Apparatus

Next, operation of the above-described control apparatus 3 will be described.

Hereinafter, as operation of the control apparatus 3, operation of restricting an output value to be supplied (energized) to the heating element 922 in accordance with the state of the pair of lead wire connecting units 9221 (equivalent to a method of operation of the thermal energy treatment apparatus according to the present disclosure) will be mainly described.

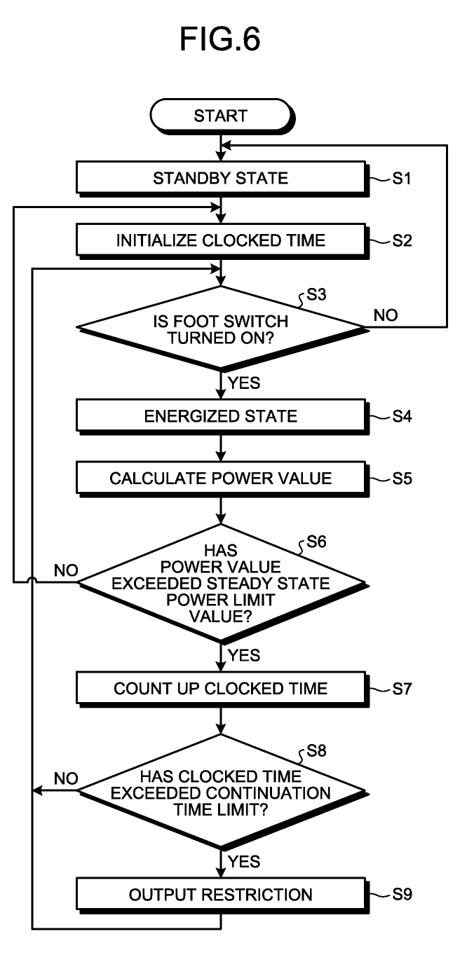

FIG. 6 is a flowchart illustrating operation of the control apparatus 3.

When the power supply (not illustrated) of the thermal energy treatment apparatus 1 is turned on by the operator, the energization controller 331 sets the energy treatment tool 2 to a standby state (step S1).

After step S1, the power value determination unit 3321 initializes the clocked time (step S2).

After step S2, the control unit 33 determines whether the foot switch 4 has been turned on (step S3).

When the foot switch 4 is turned off (step S3: No), the control apparatus 3 returns to step S1.

In contrast, when the foot switch 4 is turned on (step S3: Yes), the energization controller 331 switches the energy treatment tool 2 to the energized state (step S4: energization step). Then, while grasping the temperature of the heat transfer plate 91, the energization controller 331 executes feedback control of the heating element 922 (control of an output value (power value) to be supplied (energized) to the heating element 922) so as to set the heat transfer plate 91 to a target temperature.

After step S4, the power value determination unit 3321 calculates a power value being supplied (energized) to the heating element 922 based on the current value and the voltage value detected by the sensor 32 (step S5).

After step S5, the power value determination unit 3321 compares the power value calculated in step S5 with the steady state power limit value, and determines whether the power value has exceeded the steady state power limit value (step S6).

When it is determined that the power value has not exceeded the steady state power limit value (step S6: No), the control apparatus 3 returns to step S2.

In contrast, when it is determined that the power value has exceeded the steady state power limit value (step S6: Yes), the power value determination unit 3321 counts up the clocked time (step S7).

After step S7, the time determination unit 3322 compares the clocked time counted up in step S6 with the continuation time limit, and determines whether the clocked time has exceeded the continuation time limit (step S8).

The above-described steps S5 to S8 correspond to a state determination step according to the present disclosure.

When it is determined that the clocked time has not exceeded the continuation time limit (step S8: No), the control apparatus 3 returns to step S3.

In contrast, when it is determined that the clocked time has exceeded the continuation time limit (step S8: Yes), the output restriction unit 333 controls operation of the thermal energy output unit 31 to restrict an output value to be supplied (energized) to the heating element 922 (output restriction) (step S9: output restriction step). Thereafter, the control apparatus 3 returns to step S3.

The output restriction of step S9 is executed until the foot switch 4 is turned off at step S3 (step S3: No) and switched to the standby state (step S1). That is, after step S9, output restriction is constantly executed during repetitive execution of steps S3 to S9.

Specific Example of Waveform of Power Value

Next, a specific example of a waveform of the power value output to the heating element 922 by the above-described operation of the control apparatus 3 will be described.

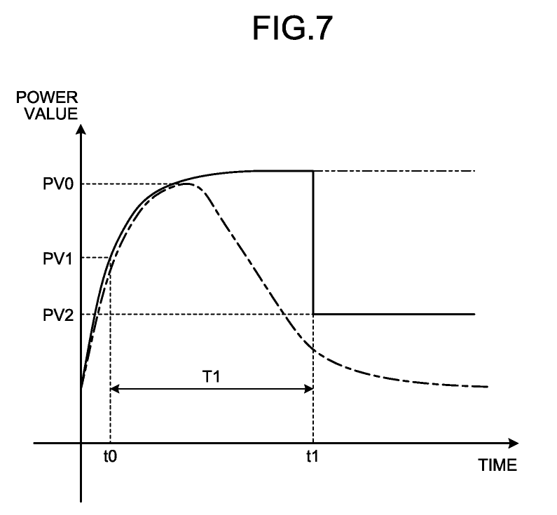

FIG. 7 is a diagram illustrating an example of a waveform of a power value output to the heating element 922 by the operation of the control apparatus 3. Specifically, the waveform illustrated by the solid line in FIG. 7 indicates a waveform of a power value when treatment (treatment on an organ having extremely large heat capacity because of a large amount of water, treatment in an environment having high likelihood of heat dissipation such as in blood) has been performed in an environment with a large heat capacity. Moreover, a waveform indicated by the two-dot chain line in FIG. 7 illustrates a waveform of a power value when the output restriction (step S9) is not performed in the treatment in an environment with a large heat capacity. Furthermore, a waveform indicated by a one-dot chain line in FIG. 7 illustrates a waveform of a power value when the treatment is performed in a normal environment, not in an environment with a large heat capacity.

Hereinafter, a case where treatment is performed in a normal environment and a case where treatment is performed in an environment with large heat capacity will be sequentially described.

Case where Treatment is Performed in Normal Environment

When the treatment is performed in a normal environment, when the feedback control of the heating element 922 is started (step S4), a large amount of power (power value PV0 (peak value)) is supplied (energized) to the heating element 922 at an initial stage in order to enable the heat transfer plate 91 to reach a target temperature at a high speed as indicated by the one-dot chain line in FIG. 7. Then, after enabling the heat transfer plate 91 to reach the target temperature, the power below the power value PV0 is supplied (energized) because it is sufficient to supply (energize) power needed to hold the temperature, to the heating element 922.

When the treatment is performed in such a normal environment, the power value (for example, power value PV0) supplied (energized) to the heating element 922 at the initial stage exceeds a steady state power limit value PV1 (step S6: Yes). Then, at the timing t0 when the power value exceeds the steady state power limit value PV1, clocking is started (step S7). The clocked time, however, does not exceed a continuation time limit T1 (step S8: No), and thus, the output restriction (step S9) is not performed.

Case where Treatment is Performed in Environment with Large Heat Capacity

When the treatment is performed in an environment with large heat capacity, when the feedback control of the heating element 922 is started (step S4), a large amount of power is supplied (energized) to the heating element 922 in order to enable the heat transfer plate 91 to reach a target temperature at a high speed as indicated by the solid line in FIG. 7, similarly to the case of treatment in a normal environment as described above.

Note that when treatment is performed in an environment with a large heat capacity, the heat transfer plate 91 is immersed in moisture, blood, or the like, and thus the heat transfer plate 91 cools unless heating is continued at high power output. Therefore, as indicated by the two-dot chain line in FIG. 7, a large power is supplied (energized) to the heating element 922 even after the heat transfer plate 91 reaches the target temperature.

When the treatment is performed in such an environment where the heat capacity is large, the power value supplied (energized) to the heating element 922 at the initial stage exceeds the steady state power limit value PV1 (step S6: Yes), similarly to the case of treatment in a normal environment. Moreover, at the timing t0 when the power value exceeds the steady state power limit value PV1, clocking is started (step S7). Subsequently, the power value is maintained to result in the clocked time exceeding the continuation time limit T1 (step S8: Yes). Therefore, the output value (power value) supplied (energized) to the heating element 922 at the timing t1 at which the clocked time exceeds the continuation time limit T1 is restricted (output restriction) to a safe power value PV2 (power value smaller than the steady state power limit value PV1) (step S9).

Note that this output restriction is sufficient to reduce the output value supplied (energized) to the heating element 922, and thus, it is allowable to restrict the power to the safe power value PV2, or stop energization to the heating element 922 (setting output value (power value) to zero).

In the thermal energy treatment apparatus 1 according to the first embodiment described above, the state of the pair of lead wire connecting units 9221 is determined based on the index value of the temperature of the pair of lead wire connecting units 9221, and the output value of energization of the heating element 922 is restricted based on a result of determination.

This enables determination of whether there is a possibility of an occurrence of an overheated state of the pair of lead wire connecting units 9221. Then, when it is determined that there is a possibility of an occurrence of an overheated state of the pair of lead wire connecting units 9221, it is possible to avoid the occurrence of an overheated state of the pair of lead wire connecting units 9221 by restricting the output value of energization of the heating element 922.

In particular, in the thermal energy treatment apparatus 1 according to the first embodiment, the power value supplied (energized) to the heating element 922 is employed as the index value of the temperature of the pair of lead wire connecting units 9221. The thermal energy treatment apparatus 1 subsequently determines whether the power value has exceeded the steady state power limit value PV1 and whether the clocked time of continuously exceeding time has exceeded the continuation time limit.

That is, by previously grasping, by experiments or the like, the waveform of the power value (waveforms indicated by the solid line and the two-dot chain line in FIG. 7) for a case where the treatment is performed in the environment with a large heat capacity, it is possible to appropriately determine whether there is a possibility of an occurrence of an overheated state of the pair of lead wire connecting units 9221 based on the above determination.

Modification 1-1 of First Embodiment

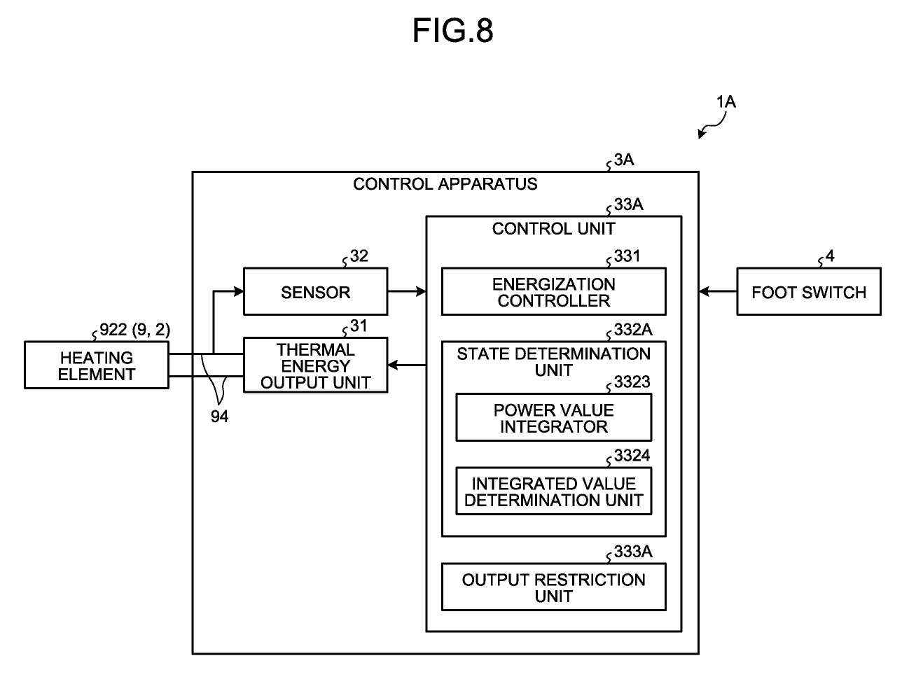

FIG. 8 is a block diagram illustrating a configuration of a control apparatus 3A constituting a thermal energy treatment apparatus 1A according to modification 1-1 of the first embodiment.

In the above-described first embodiment, a state determination unit 332A (control unit 33A) illustrated in FIG. 8 may be employed in place of the state determination unit 332 (control unit 33).

Specifically, as illustrated in FIG. 8, the state determination unit 332A includes a power value integrator 3323 and an integrated value determination unit 3324.

The power value integrator 3323 calculates the power value supplied (energized) to the heating element 922 based on the current value and the voltage value detected by the sensor 32. Then, the power value integrator 3323 sequentially integrates the calculated power values.

The integrated value determination unit 3324 compares the integrated value integrated by the power value integrator 3323 with a preset integration limit value (equivalent to a third threshold according to the present disclosure), and determines whether the integrated value has exceeded the integration limit value.

When the integrated value determination unit 3324 determines that the integrated value has exceeded the integration limit value, an output restriction unit 333A according to modification 1-1 controls operation of the thermal energy output unit 31 to restrict the output value (power value) to be supplied (energized) to the heating element 922.

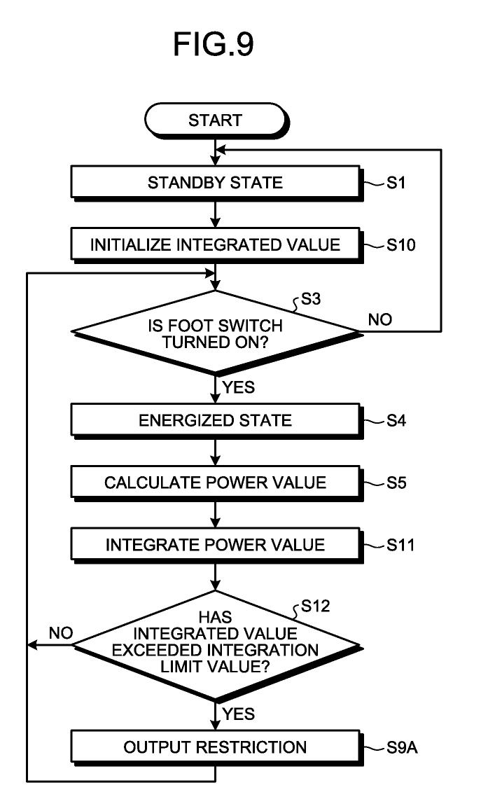

FIG. 9 is a flowchart illustrating operation of the control apparatus 3A.

As illustrated in FIG. 9, operation of the control apparatus 3A according to modification 1-1 is different from the operation (FIG. 6) of the control apparatus 3 described in the first embodiment in that steps S10 to S12 and S9A are employed in place of steps S2 and S6 to S9. Therefore, steps S10 to S12 and S9A alone will be described below.

Step S10 is executed after step S1.

Specifically, the power value integrator 3323 executes initialization of an integrated value in step S10.

Step S11 is executed after step S5.

Specifically, in step S11, the power value integrator 3323 sequentially integrates the power values calculated in step S5.

After step S11, the integrated value determination unit 3324 compares the integrated value integrated in step S11 with the integration limit value, and determines whether the integrated value has exceeded the integration limit value (step S12).

Note that steps S5, S11, and S12 correspond to the state determination step according to the present disclosure.

When it is determined that the integrated value has not exceeded the integration limit value (step S12: No), the control apparatus 3A returns to step S3.

In contrast, when it is determined that the integrated value exceeds the integration limit value (step S12: Yes), the output restriction unit 333A controls operation of the thermal energy output unit 31 to execute output restriction similarly to step S9 described in the above first embodiment (step S9A: output restriction step). Thereafter, the control apparatus 3A returns to step S3.

Note that the output restriction of step S9A is executed until the foot switch 4 is turned off at step S3 (step S3: No) and switched to the standby state (step S1), similarly to the above-described first embodiment.

Next, a specific example of a waveform of the power value output to the heating element 922 by the above-described operation of the control apparatus 3A will be described.

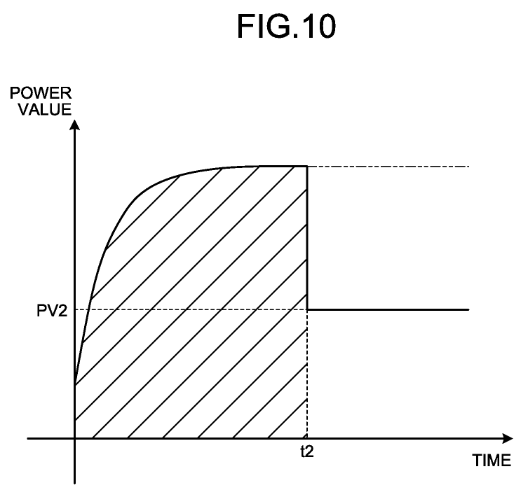

FIG. 10 is a diagram illustrating an example of a waveform of a power value output to the heating element 922 by the operation of the control apparatus 3A. Specifically, FIG. 10 is a diagram corresponding to FIG. 7. Note that the waveform of the power value when the treatment is performed in a normal environment has the same waveform as the waveform described in the first embodiment (indicated by a one-dot chain line in FIG. 7). For this reason, in FIG. 10, illustration of the waveform of the power value in the case where treatment is performed in the normal environment is omitted.

In modification 1-1, after the energy treatment tool 2 is switched to the normal state (step S4), calculation and integration of power values (steps S5 and S6) are started (the state of performing integration is hatched in FIG. 10). Subsequently, at the timing t2 when the integrated value exceeds the integration limit value (step S12: Yes), the output value (power value) to be supplied (energized) to the heating element 922 is restricted (output restriction) to the safety power value PV2 (Step S9A).

That is, even when the output restriction is performed based on the integrated value of the power values as in modification 1-1, the waveform of the power value when the treatment is performed in an environment with a large heat capacity is substantially the same waveform as described in the above first embodiment (indicated by the solid line in FIG. 7).

With the thermal energy treatment apparatus 1A according to modification 1-1 described above, the following effects would be achieved in addition to the effects similar to the effects in the above-described first embodiment.

With the thermal energy treatment apparatus 1A according to modification 1-1, even without clocking the clocked time during which the power value continuously exceeds the steady state power limit value PV1 as in the above-described first embodiment, it is possible to execute the output restriction in accordance with the integration of the power value since the integrated value of the power value includes the concept of the clocking. Therefore, by omitting the clocking, it is possible to reduce the processing load of the control unit 33A (state determination unit 332A).

Modification 1-2 of First Embodiment

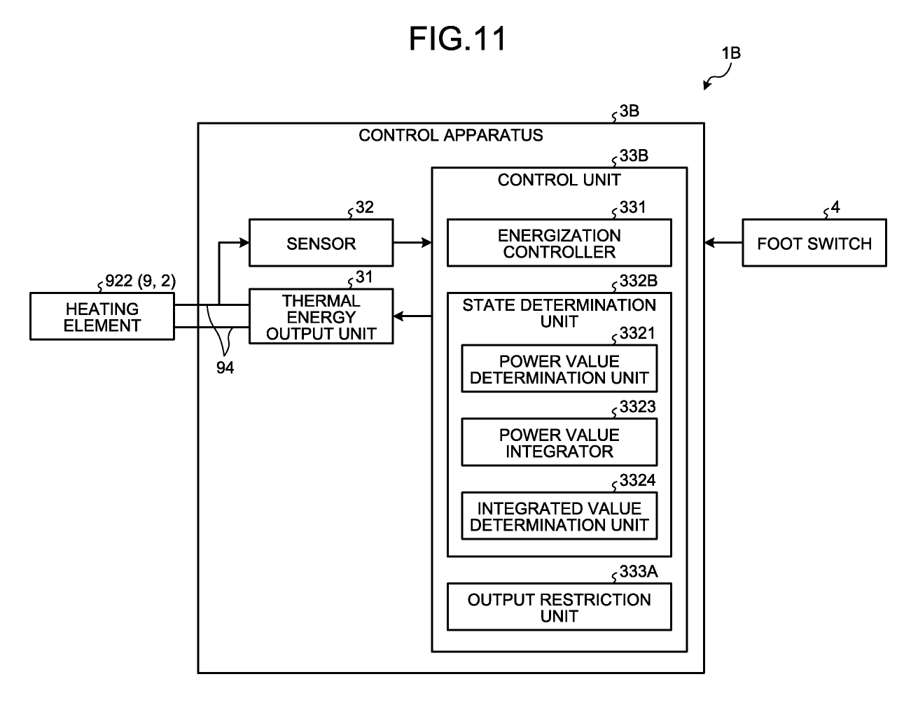

FIG. 11 is a block diagram illustrating a configuration of a control apparatus 3B constituting a thermal energy treatment apparatus 1B according to modification 1-2 of the first embodiment.

In the above-described first embodiment, a state determination unit 332B (control unit 33B) illustrated in FIG. 11 may be employed in place of the state determination unit 332 (control unit 33).

Specifically, as illustrated in FIG. 11, the state determination unit 332B includes the power value determination unit 3321 described in the above-described first embodiment, and the power value integrator 3323 and the integrated value determination unit 3324 described in the above modification 1-1.

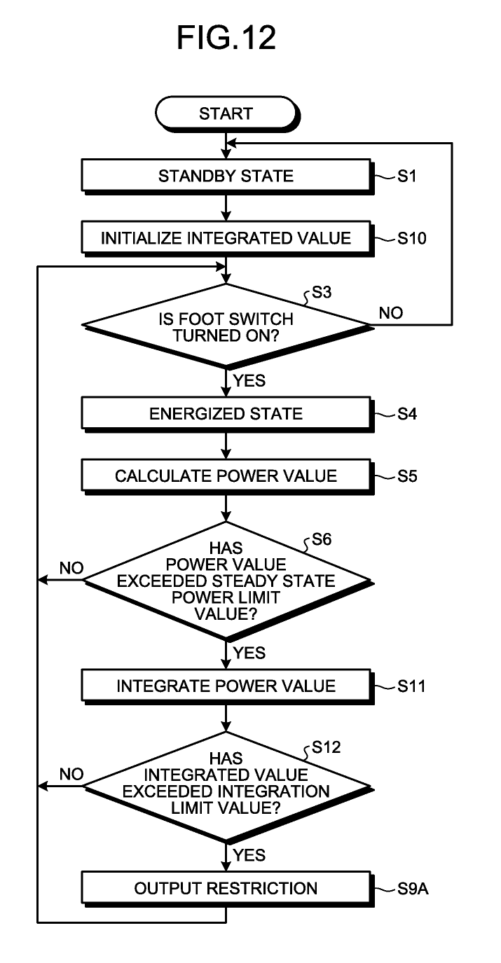

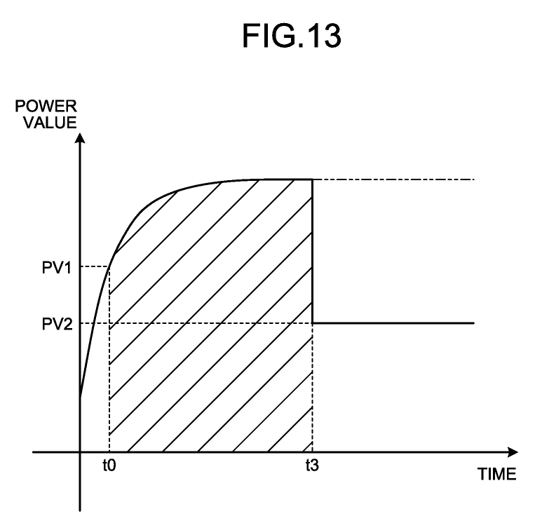

FIG. 12 is a flowchart illustrating operation of the control apparatus 3B. FIG. 13 is a diagram illustrating an example of a waveform of a power value output to the heating element 922 by the operation of the control apparatus 3B. Specifically, FIG. 13 is a diagram corresponding to FIGS. 7 and 10. Note that the waveform of the power value when the treatment is performed in a normal environment has the same waveform as the waveform described in the first embodiment (indicated by a one-dot chain line in FIG. 7). For this reason, in FIG. 13, illustration of the waveform of the power value in the case where treatment is performed in the normal environment is omitted.

As illustrated in FIG. 12, operation of the control apparatus 3B according to modification 1-2 is different from the operation (FIG. 9) of the control apparatus 3A described in modification 1-1 in that step S6 described in the first embodiment is added.

Specifically, step S6 is executed between step S5 and step S11. That is, in this modification 1-2, as illustrated in FIG. 13, at the timing t0 when the power value calculated in step S5 exceeds the steady state power limit value PV1 (step S6: Yes), integration of the power value is started (step S11) (the state of performing integration is hatched in FIG. 13). Subsequently, at the timing t3 when the integrated value exceeds the integration limit value (step S12: Yes), the output value (power value) to be supplied (energized) to the heating element 922 is restricted (output restriction) to the safety power value PV2 (Step S9A). When the power value calculated in step S5 has not exceeded the steady state power limit value PV1 (step S6: No), the processing returns to step S3.

Note that steps S5, S6, S11, and S12 correspond to the state determination step according to the present disclosure.

With the thermal energy treatment apparatus 1B according to modification 1-2 described above, the following effects would be achieved in addition to the effects similar to the effects in the above-described modification 1-1.

The thermal energy treatment apparatus 1B according to modification 1-2 starts integration of the power value after the power value exceeds the steady state power limit value PV1. Therefore, compared to the first embodiment described above, when the power value after exceeding the steady state power limit value PV1 is relatively large, the output restriction may be executed more quickly. Conversely, when the power value after exceeding the steady state power limit value PV1 is relatively small, the output restriction is not executed and the operator may use the power for a longer time.

Modification 1-3 of First Embodiment

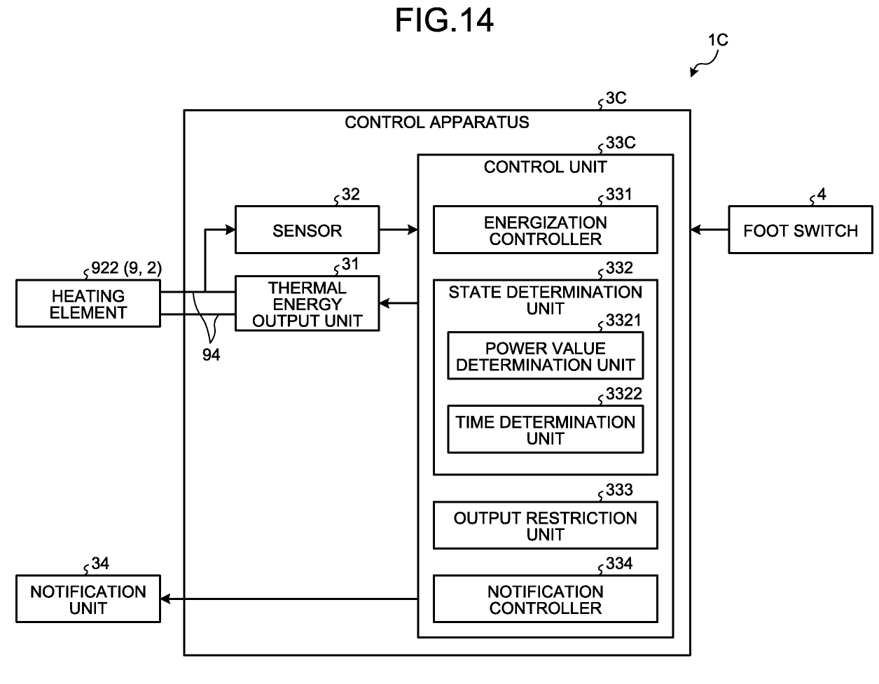

FIG. 14 is a block diagram illustrating a configuration of a control apparatus 3C constituting a thermal energy treatment apparatus 1C according to modification 1-3 of the first embodiment.

In the above-described first embodiment, it is allowable to employ a notification unit 34 and employ a control unit 33C to which a notification controller 334 is added.

Specifically, the notification unit 34 notifies predetermined information. Examples of the notification unit 34 may include a display to display predetermined information, a light emitting diode (LED) to notify predetermined information by lighting or blinking, and a speaker to notify predetermined information by sound.

When the output restriction unit 333 executes the output restriction, the notification controller 334 activates the notification unit 34 to notify that the output restriction is being executed.

With the thermal energy treatment apparatus 1C according to modification 1-3 described above, the following effects would be achieved in addition to the effects similar to the effects in the above-described first embodiment.

That is, with the thermal energy treatment apparatus 1C according to modification 1-3, it is possible to give the operator recognition that output restriction is being executed by the operation of the notification unit 34.

### Second Embodiment

Next, a second embodiment will be described.

In the following description, the same reference numerals are given to similar configurations as those of the above-described first embodiment, and a detailed description thereof will be omitted or simplified.

In the first embodiment described above, the power value supplied (energized) to the heating element 922 is employed as an index value according to the present disclosure.

In contrast, in the second embodiment, a temperature of the pair of lead wire connecting units 9221 is employed as the index value according to the present disclosure.

Hereinafter, a configuration of a thermal energy treatment apparatus and operation of a control apparatus according to the second embodiment will be sequentially described.

Configuration of Thermal Energy Treatment Apparatus

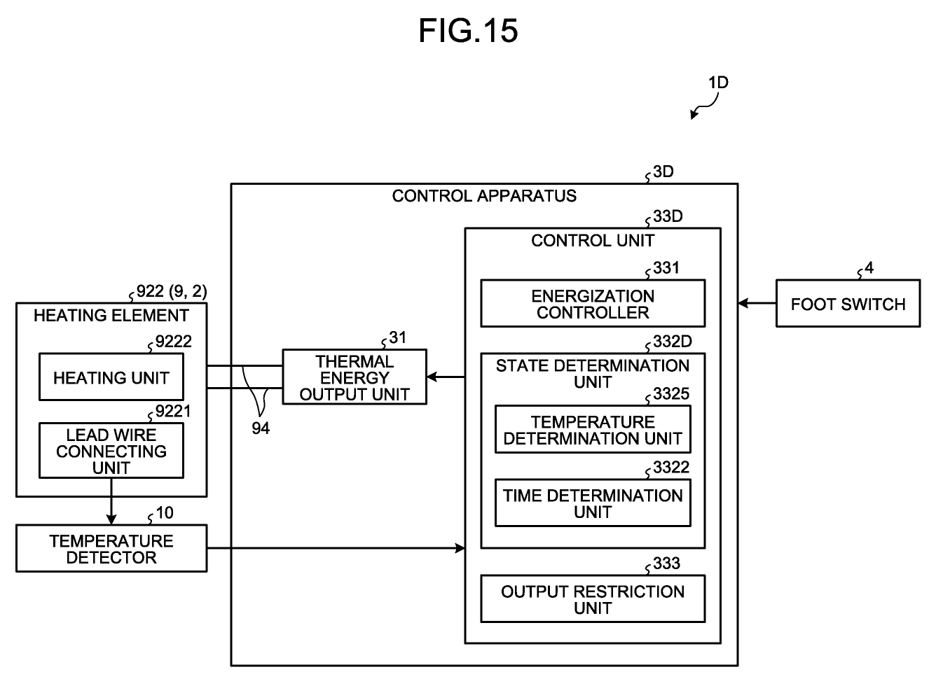

FIG. 15 is a block diagram illustrating a configuration of a control apparatus 3D constituting a thermal energy treatment apparatus 1D according to the second embodiment.

As illustrated in FIG. 15, compared to the thermal energy treatment apparatus 1 (FIG. 5) described in the above-described first embodiment, the thermal energy treatment apparatus 1D further includes a temperature detector 10 and employs the control apparatus 3D having functions partially changed from the control apparatus 3.

The temperature detector 10 is a temperature sensor constituted with a thermocouple, a thermistor, or the like, and detects the temperature of the pair of lead wire connecting units 9221. For example, as a configuration to locate the temperature detector 10, it is possible to employ a configuration in which the temperature detector 10 is directly attached to the pair of lead wire connecting units 9221 or a configuration in which the temperature detector 10 is on the other surface of the insulating substrate 921 (surface on which the heating element 922 is not provided), specifically, at a position facing the pair of lead wire connecting units 9221. The temperature detector 10 subsequently outputs a signal corresponding to the detected temperature to the control apparatus 3D.

As illustrated in FIG. 15, compared to the control apparatus 3 (FIG. 5) described in the above-described first embodiment, the control apparatus 3D omits the sensor 32 and employs a state determination unit 332D (control unit 33D) in place of the state determination unit 332 (the control unit 33).

Note that while FIG. 15 omits the sensor 32, when the temperature of the heat transfer plate 91 is to be calculated based on the current value and the voltage value detected by the sensor 32 in the feedback control of the heating element 922 (control of the output value supplied (energized) to the heating element 922), there is no need to omit the sensor 32.

As illustrated in FIG. 15, the state determination unit 332D further includes a temperature determination unit 3325 in addition to the time determination unit 3322 described in the first embodiment.

The temperature determination unit 3325 compares the temperature of the pair of lead wire connecting units 9221 detected by the temperature detector 10 (hereinafter referred to as detected temperature) with a preset temperature limit value (equivalent to the first threshold according to the present disclosure), and clocks the time (hereinafter referred to as clocked time) during which the detected temperature continuously exceeds the temperature limit value. That is, the temperature determination unit 3325 has a function as an index value determination unit according to the present disclosure.

Operation of Control Apparatus

Next, operation of the above-described control apparatus 3D will be described.

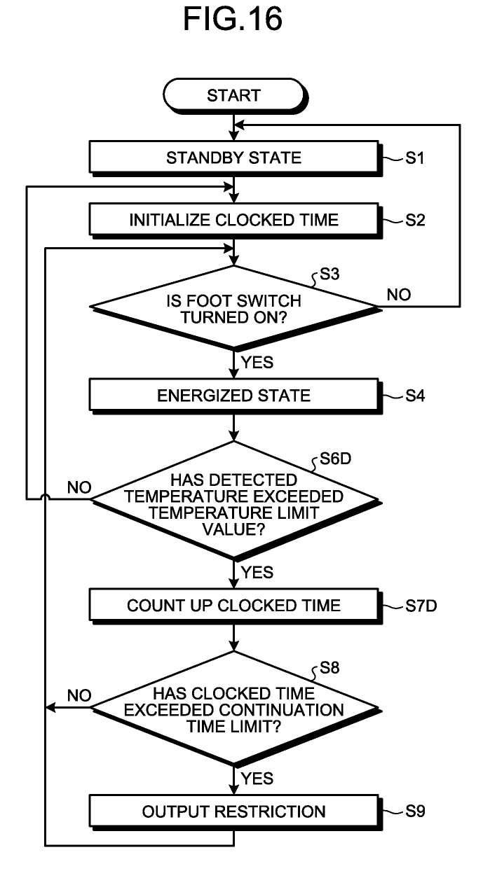

FIG. 16 is a flowchart illustrating operation of the control apparatus 3D.

As illustrated in FIG. 16, operation of the control apparatus 3D according to the second embodiment is different from the operation (FIG. 6) of the control apparatus 3 described in the first embodiment in that step S5 is omitted and steps S6D and S7D are employed in place of steps S6 and S7. Therefore, step S6D alone will be described below.

Step S6D is executed after step S4.

Specifically, in step S6D, the temperature determination unit 3325 compares the detected temperature detected by the temperature detector 10 with the temperature limit value, and determines whether the detected temperature has exceeded the temperature limit value.

When it is determined that the detected temperature has not exceeded the temperature limit value (step S6D: No), the control apparatus 3D returns to step S2.

In contrast, when it is determined that the detected temperature exceeds the temperature limit value (step S6D: Yes), the temperature determination unit 3325 counts up the clocked time (step S7D). Thereafter, the control apparatus 3D moves to step S8.

The steps S6D, S7D, and S8 correspond to the state determination step according to the present disclosure.

With the thermal energy treatment apparatus 1D according to the second embodiment described above, the following effects would be achieved in addition to the effects similar to the effects in the above-described first embodiment.

In the thermal energy treatment apparatus 1D according to the second embodiment, “the detected temperature (temperature of the pair of lead wire connecting units 9221) detected by the temperature detector 10” is employed as the index value according to the present disclosure.

This enables reliable determination of whether there is a possibility of an occurrence of an overheated state of the pair of lead wire connecting units 9221.

Modification 2-1 of Second Embodiment

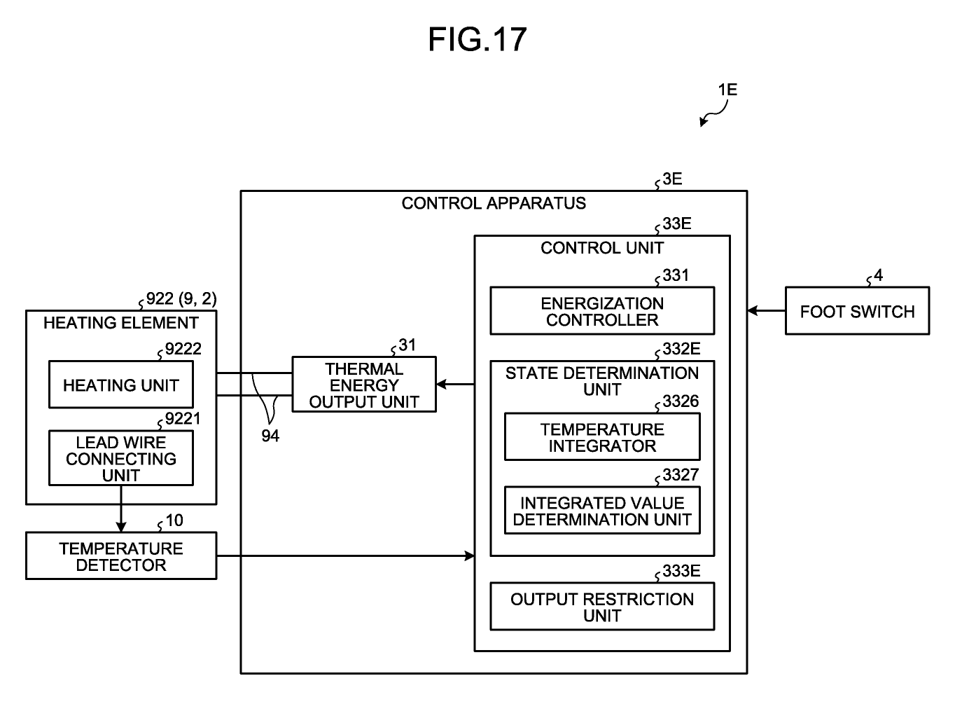

FIG. 17 is a block diagram illustrating a configuration of a control apparatus 3E constituting a thermal energy treatment apparatus 1E according to modification 2-1 of the second embodiment.

In the above-described second embodiment, a state determination unit 332E (control unit 33E) illustrated in FIG. 17 may be employed in place of the state determination unit 332D (control unit 33D).

Specifically, as illustrated in FIG. 15, the state determination unit 332E includes a temperature integrator 3326 and an integrated value determination unit 3327.

The temperature integrator 3326 sequentially integrates the detected temperatures detected by the temperature detector 10.

The integrated value determination unit 3327 compares the integrated value integrated by the temperature integrator 3326 with a preset integration limit value (equivalent to a third threshold according to the present disclosure), and determines whether the integrated value has exceeded the integration limit value.

When the integrated value determination unit 3327 determines that the integrated value has exceeded the integration limit value, an output restriction unit 333E according to modification 2-1 controls operation of the thermal energy output unit 31 to restrict the output value (power value) to be supplied (energized) to the heating element 922.

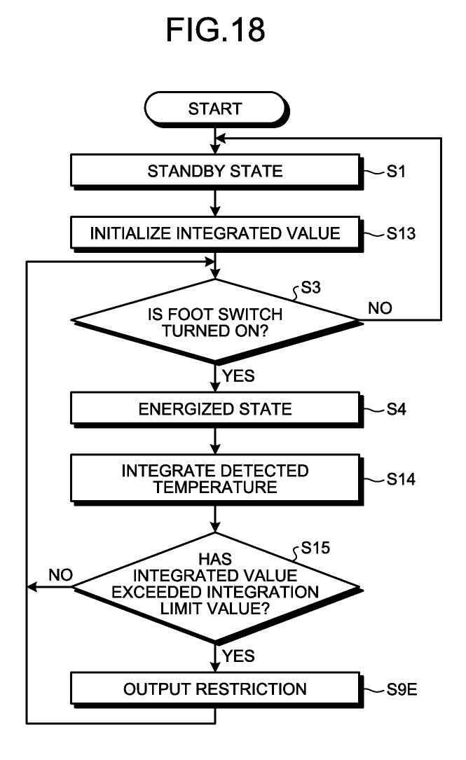

FIG. 18 is a flowchart illustrating operation of the control apparatus 3E.

As illustrated in FIG. 18, operation of the control apparatus 3E according to modification 2-1 is different from the operation (FIG. 16) of the control apparatus 3D described in the second embodiment in that steps S13 to S15 and S9E are employed in place of steps S2 and S6D, S7D, S8, and S9. Therefore, steps S13 to S15 and S9E alone will be described below.

Step S13 is executed after step S1.

Specifically, the temperature integrator 3326 initializes the integrated value in step S13.

Step S14 is executed after step S4.

Specifically, in step S14, the temperature integrator 3326 sequentially integrates the detected temperatures detected by the temperature detector 10.

After step S14, the integrated value determination unit 3327 compares the integrated value integrated in step S14 with the integration limit value, and determines whether the integrated value exceeds the integration limit value (step S15).

Steps S14 and S15 correspond to the state determination step according to the present disclosure.

When it is determined that the integrated value has not exceeded the integration limit value (step S15: No), the control apparatus 3E returns to step S3.

In contrast, when it is determined that the integrated value has exceeded the integration limit value (step S15: Yes), the output restriction unit 333E controls operation of the thermal energy output unit 31 to execute output restriction (step S9E: output restriction step) similarly to step S9 described in the above first embodiment. Thereafter, the control apparatus 3E returns to step S3.

Note that the output restriction of step S9E is executed until the foot switch 4 is turned off at step S3 (step S3: No) and switched to the standby state (step S1), similarly to the above-described first and second embodiments.

With the thermal energy treatment apparatus 1E according to modification 2-1 described above, effects similar to the effects in the above-described second embodiment and modification 1-1 would be achieved.

Modification 2-2 of Second Embodiment

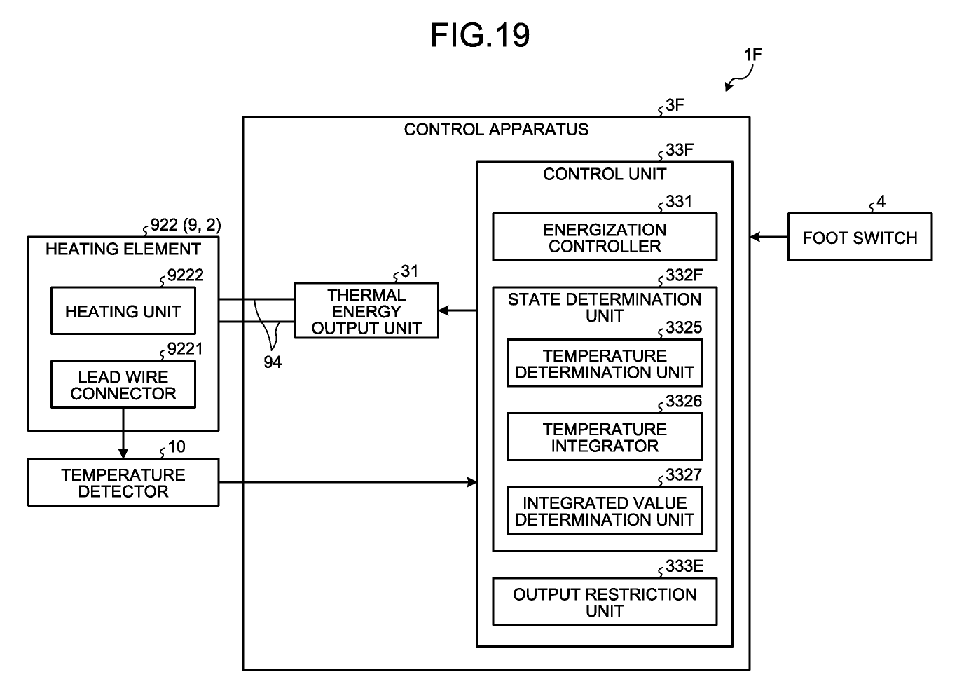

FIG. 19 is a block diagram illustrating a configuration of a control apparatus 3F constituting a thermal energy treatment apparatus 1F according to modification 2-2 of the second embodiment.

In the above-described second embodiment, a state determination unit 332F (control unit 33F) illustrated in FIG. 19 may be employed in place of the state determination unit 332D (control unit 33D).

Specifically, as illustrated in FIG. 19, the state determination unit 332F includes the temperature determination unit 3325 described in the above-described second embodiment, and the temperature integrator 3326 and the integrated value determination unit 3327 described in the above modification 2-1.

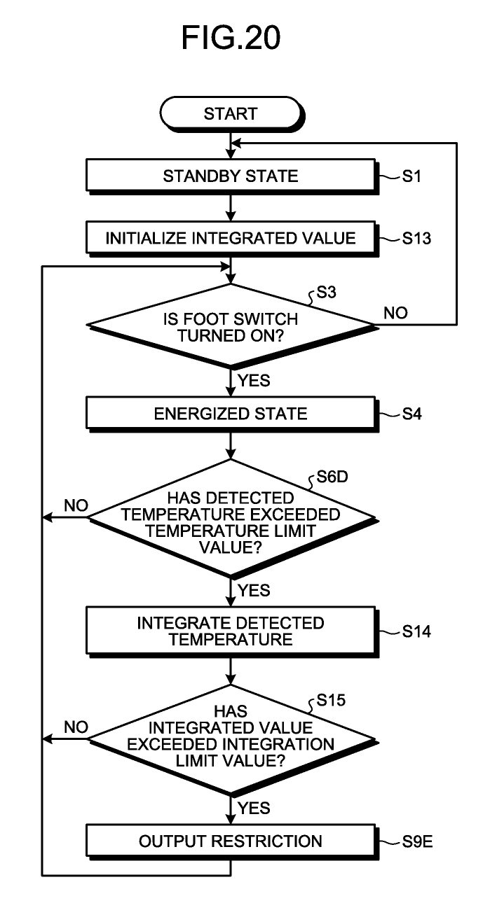

FIG. 20 is a flowchart illustrating operation of the control apparatus 3F.

As illustrated in FIG. 20, operation of the control apparatus 3F according to modification 2-2 is different from the operation (FIG. 18) of the control apparatus 3E described in modification 2-1 in that step S6D described in the second embodiment is added.

Specifically, step S6D is executed between step S4 and step S14. That is, in modification 2-2, integration of the detected temperature (step S14) is started at a timing when the detected temperature exceeds the temperature limit value (step S6D: Yes). Subsequently, at a timing when the integrated value exceeds the integration limit value (step S15: Yes), the output value (power value) to be supplied (energized) to the heating element 922 is restricted (output restriction) (step S9E). When the detected temperature has not exceeded the temperature limit value (step S6D: No), the processing returns to step S3.

Steps S6D, S14, and S15 correspond to the state determination step according to the present disclosure.

With the thermal energy treatment apparatus 1F according to modification 2-2 described above, effects similar to the effects in the above-described second embodiment and modification 1-2 would be achieved.

### Third Embodiment

Next, a third embodiment will be described.

In the following description, the same reference numerals are given to similar configurations as those of the above-described first embodiment, and a detailed description thereof will be omitted or simplified.

In the first embodiment described above, the power value supplied (energized) to the heating element 922 is employed as an index value according to the present disclosure.

In contrast, in the third embodiment, an impedance of the heating element 922 in a state of being energized with alternating current to the heating element 922 is employed as the index value according to the present disclosure.

Hereinafter, a configuration of a thermal energy treatment apparatus and operation of a control apparatus according to the third embodiment will be sequentially described.

Configuration of Thermal Energy Treatment Apparatus

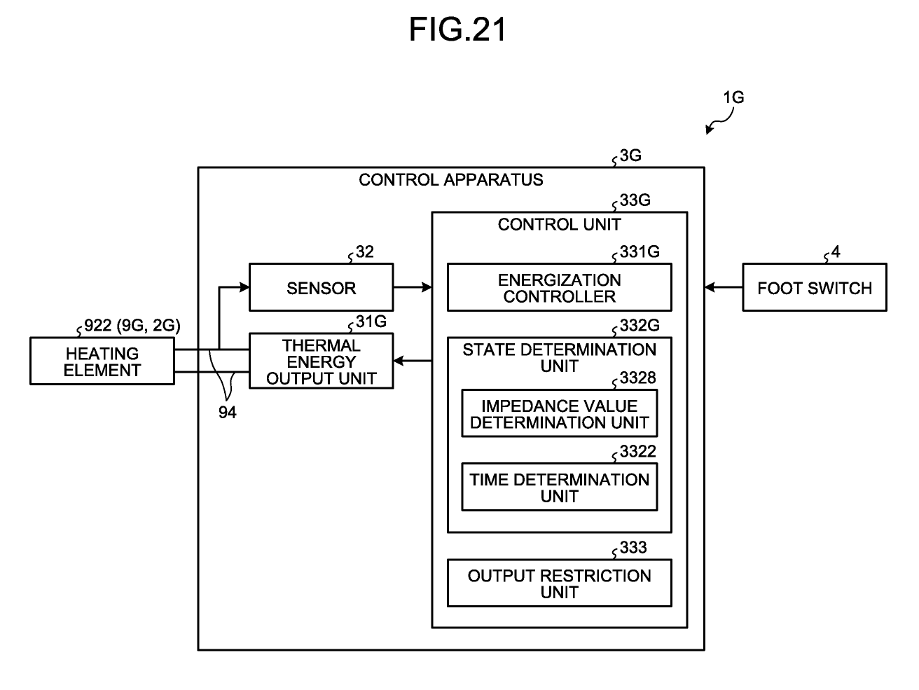

FIG. 21 is a block diagram illustrating a configuration of a control apparatus 3G constituting a thermal energy treatment apparatus 1G according to the third embodiment.

As illustrated in FIG. 21, compared to the thermal energy treatment apparatus 1 (FIG. 5) described in the above-mentioned first embodiment, the thermal energy treatment apparatus 1G employs a therapeutic energy applying structure 9G (energy treatment tool 2G) having configurations partially changed from the therapeutic energy applying structure 9 (energy treatment tool 2), and employs a control apparatus 3G having functions partially changed from the control apparatus 3.

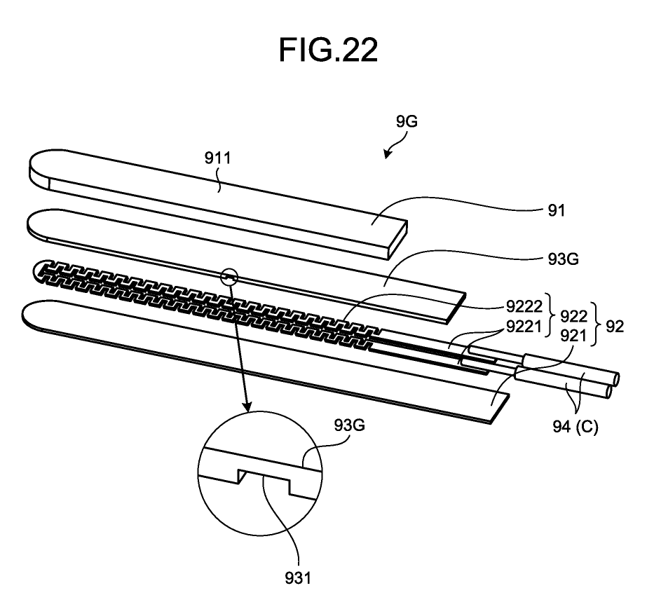

FIG. 22 is a diagram illustrating the therapeutic energy applying structure 9G.

As illustrated in FIG. 22, compared to the therapeutic energy applying structure 9 (FIG. 4) described in the first embodiment, the therapeutic energy applying structure 9G employs an adhesive sheet 93G in which a recess 931 is formed on a flexible substrate 92-side surface of the adhesive sheet 93.

The recess 931 is provided at a position facing the heating unit 9222 and is formed so as to penetrate through both ends of the adhesive sheet 93G in the width direction.

That is, in the third embodiment, the therapeutic energy applying structure 9G is configured such that when a distal end portion of the energy treatment tool 2G is immersed in a liquid, the liquid comes in contact with the heating unit 9222 via the recess 931. Note that, as long as the structure allows the liquid to come into contact with the heating unit 9222, the recess 931 is not necessarily provided in the adhesive sheet 93G, and a recess similar to the recess 931 may be provided in the insulating substrate 921. Moreover, the present disclosure is not limited to the structure having a recess, and the material of the adhesive sheet 93G and the insulating substrate 921 may be a material that may permeate or infiltrate the liquid.

As illustrated in FIG. 21, compared to the control apparatus 3 (FIG. 5) described in the first embodiment, the control apparatus 3G employs a thermal energy output unit 31G in place of the thermal energy output unit 31 and employs a control unit 33G having functions partially changed from the control unit 33.

As illustrated in FIG. 21, the control unit 33G further includes an energization controller 331G and a state determination unit 332G in addition to the output restriction unit 333 described in the first embodiment.

Compared to the energization controller 331 and the thermal energy output unit 31 (configuration of energizing the heating element 922 with direct current) described in the first embodiment, the energization controller 331G and the thermal energy output unit 31G are configured to energize the heating element 922 with alternating current (for example, a high frequency of 20 kHz or more) to cause the heating element 922 to generate heat with the alternating current energization (execute feedback control of the heating element 922 by alternating current energization).

As illustrated in FIG. 21, the state determination unit 332G further includes an impedance value determination unit 3328 in addition to the time determination unit 3322 described in the first embodiment.

The impedance value determination unit 3328 calculates an impedance value of the heating element 922 in a state where the heating element 922 is energized with alternating current based on the current value and the voltage value detected by the sensor 32. Subsequently, the impedance value determination unit 3328 compares the calculated impedance value with a preset impedance limit value (equivalent to a fourth threshold according to the present disclosure), and clocks the time during which the impedance value is continuously below the impedance limit value (hereinafter referred to as “clocked time”). That is, the sensor 32 according to the third embodiment has a function as a second detection unit according to the present disclosure.

Operation of Control Apparatus

Next, operation of the above-described control apparatus 3G will be described.

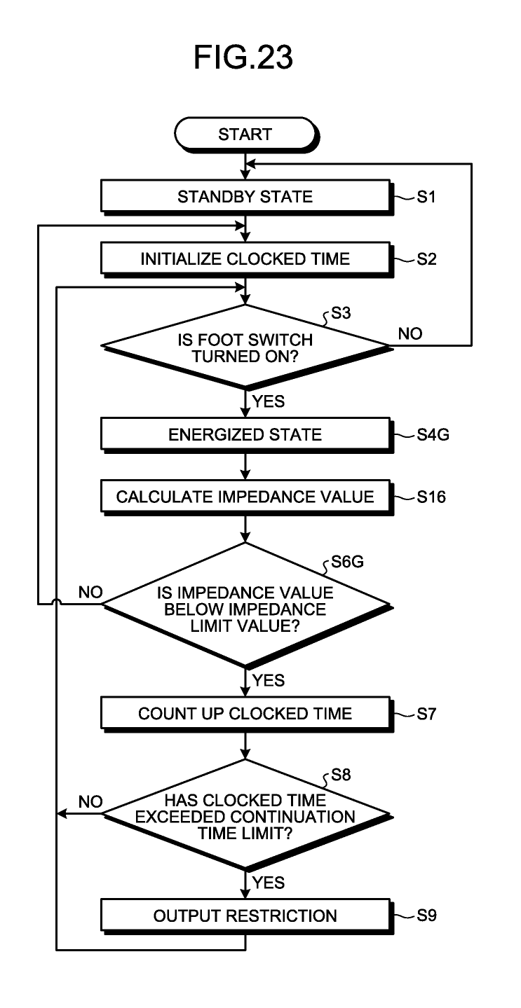

FIG. 23 is a flowchart illustrating operation of the control apparatus 3G.

As illustrated in FIG. 23, operation of the control apparatus 3G according to the third embodiment is different from the operation (FIG. 6) of the control apparatus 3 described in the first embodiment in that step S5 is omitted and steps S4G, S16, and S6G are employed in place of steps S4 and S6. Therefore, steps S4G, S16, S6G alone will be described below.

Step S4G (energization step) is executed when the foot switch 4 is turned on in step S3 (step S3: Yes).

Note that step S4G differs from step S4 described in the first embodiment in that the heating element 922 is energized with alternating current.

After step S4G, the impedance value determination unit 3328 calculates an impedance value of the heating element 922 (step S16) in a state where the heating element 922 is energized with alternating current based on the current value and the voltage value detected by the sensor 32.

After step S16, the impedance value determination unit 3328 compares the impedance value calculated in step S16 with the impedance limit value (for example, an initial value of the impedance value at the time of starting feedback control in step S4G), and determines whether the impedance value is below the impedance limit value (step S6G).

When it is determined that the impedance value is not below the impedance limit value (step S6G: No), the control apparatus 3G returns to step S2.

In contrast, when it is determined that the impedance value is below the impedance limit value (step S6G: Yes), the control apparatus 3G moves to step S7.

Steps S16, S6G, S7, and S8 correspond to the state determination step according to the present disclosure.

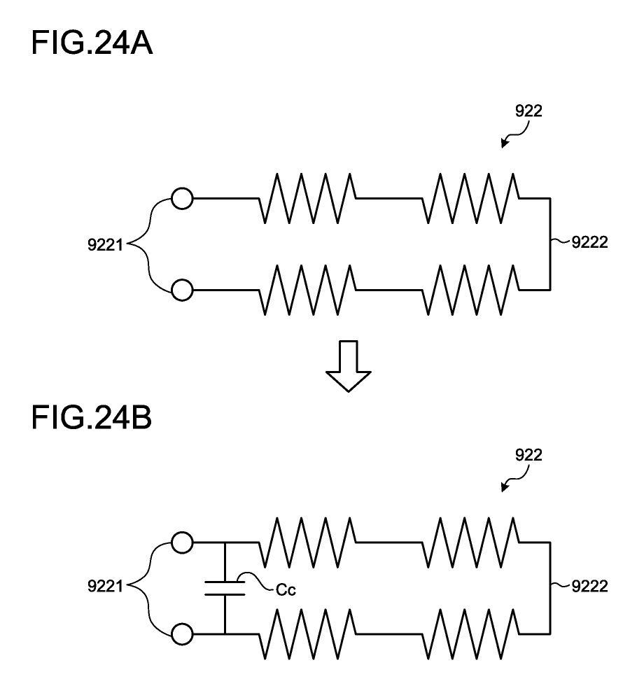

FIGS. 24A and 24B are diagrams illustrating step S6G. Specifically, FIG. 24A is a diagram illustrating a circuit model of the heating element 922 in a state where a liquid is not in contact with the heating unit 9222. FIG. 24B is a diagram illustrating a circuit model of the heating element 922 in a state where the liquid is in contact with the heating unit 9222.

Meanwhile, it is known that water has a large dielectric constant of about 80, and blood is considered to have a value close to this value. When the distal end portion of the energy treatment tool 2G is immersed in this type of liquid and the liquid comes into contact with the heating unit 9222 via the recess 931 (when the terminals of the heating element 922 are short-circuited), the impedance value changes as follows.

As illustrated in FIGS. 24A and 24B, a portion where short circuit occurred between the terminals of the heating element 922 by the liquid is to work as a capacitive component Cc (FIG. 24B). This decreases the impedance value by a level of a phase shift in comparison to the state where the liquid is not in contact with the heating unit 9222. That is, step S6G compares the impedance value with the impedance limit value (for example, the initial value of the impedance value at the time of starting feedback control at step S4G), so as to determine whether the distal end portion of the energy treatment tool 2G is immersed in the liquid (determine that it is immersed in the liquid when the impedance value is below the impedance limit value).

With the thermal energy treatment apparatus 1G according to the third embodiment described above, the following effects would be achieved in addition to the effects similar to the effects in the above-described first embodiment.

The thermal energy treatment apparatus 1G according to the third embodiment employs “the impedance value of the heating element 922 in a state where the heating element 922 is energized with alternating current” as the index value according to the present disclosure.

That is, it is possible to appropriately determine whether there is a possibility of an occurrence of an overheated state of the pair of lead wire connecting units 9221 by determining whether the distal end portion of the energy treatment tool 2G is immersed based on the impedance value of the heating element 922.

Modification 3-1 of Third Embodiment

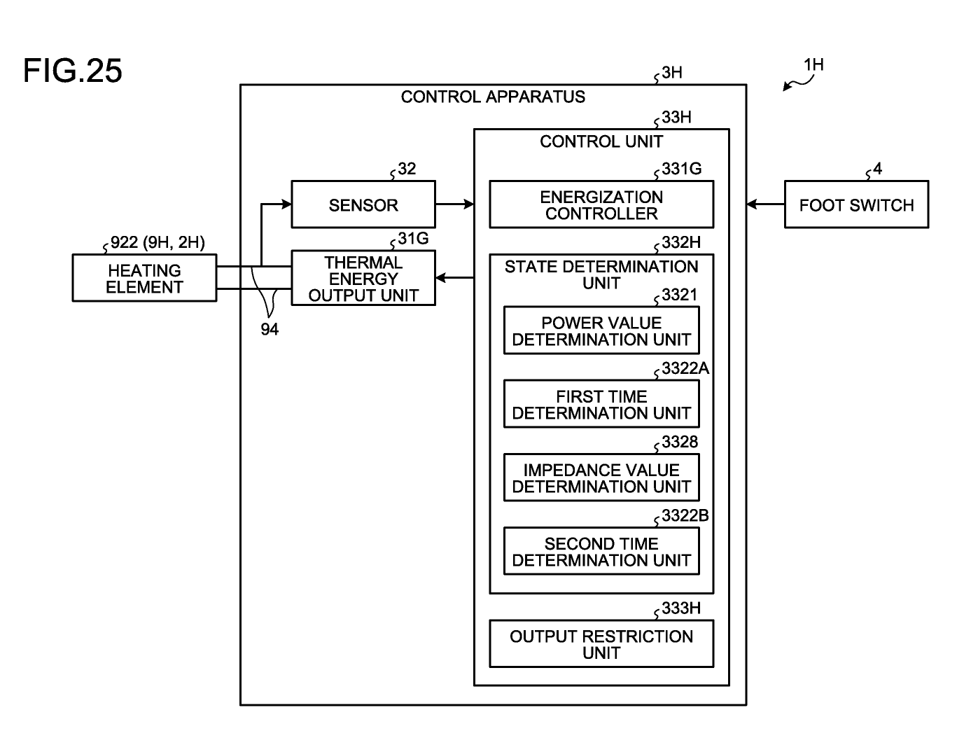

FIG. 25 is a block diagram illustrating a configuration of a control apparatus 3H constituting a thermal energy treatment apparatus 1H according to modification 3-1 of the third embodiment.

In the third embodiment described above, a therapeutic energy applying structure 9H (FIG. 25) may be employed in place of the therapeutic energy applying structure 9G, and a state determination unit 332H (control unit 33H) illustrated in FIG. 25 may be employed in place of the state determination unit 332G (control unit 33G).

Although not illustrated specifically, the therapeutic energy applying structure 9H differs from the therapeutic energy applying structure 9 (FIG. 3) described in the first embodiment merely in that the insulating member 95 is omitted (the pair of lead wire connecting units 9221 is not sealed).

That is, in modification 3-1, the therapeutic energy applying structure 9H is configured such that a liquid comes in contact with the pair of lead wire connecting units 9221 when a distal end portion of the energy treatment tool 2H is immersed in the liquid up to the position at which the pair of lead wire connecting units 9221 is arranged.

As illustrated in FIG. 25, the state determination unit 332H includes the power value determination unit 3321 described in the first embodiment, the impedance value determination unit 3328 described in the third embodiment, and first and second time determination units 3322A and 3322B similar to the time determination unit 3322 respectively described in the above-described first and third embodiments. Note that the power value determination unit 3321 according to the third embodiment has a function as an output value determination unit according to the present disclosure.

The first time determination unit 3322A compares a clocked time clocked by the power value determination unit 3321 (hereinafter referred to as a first clocked time) with a preset continuation time limit (equivalent to the second threshold according to the present disclosure, and hereinafter referred to as a first continuation time limit), and determines whether the first clocked time has exceeded the first continuation time limit. Subsequently, when it is determined that the first clocked time has exceeded the first continuation time limit, the first time determination unit 3322A sets a power limit flag (stored in a memory (not illustrated) in the control apparatus 3H) to “1” (initial value is “0”).

The second time determination unit 3322B compares a clocked time clocked by the impedance value determination unit 3328 (hereinafter referred to as a second clocked time) with a preset continuation time limit (hereinafter referred to as a second continuation time limit), and determines whether the second clocked time has exceeded the second continuation time limit. Subsequently, when it is determined that the second clocked time has exceeded the second continuation time limit, the second time determination unit 3322B sets an impedance flag (stored in a memory (not illustrated) in the control apparatus 3H) to “1” (initial value is “0”).

Subsequently, an output restriction unit 333H according to modification 3-1 reads the power limit flag and the impedance flag stored in the memory (not illustrated) in the control apparatus 3H, and controls operation of the thermal energy output unit 31 to restrict the output value (power value) to be supplied (energized) to the heating element 922 when an output restriction condition (power limit flag is “1” and the impedance flag is “0”) is satisfied.

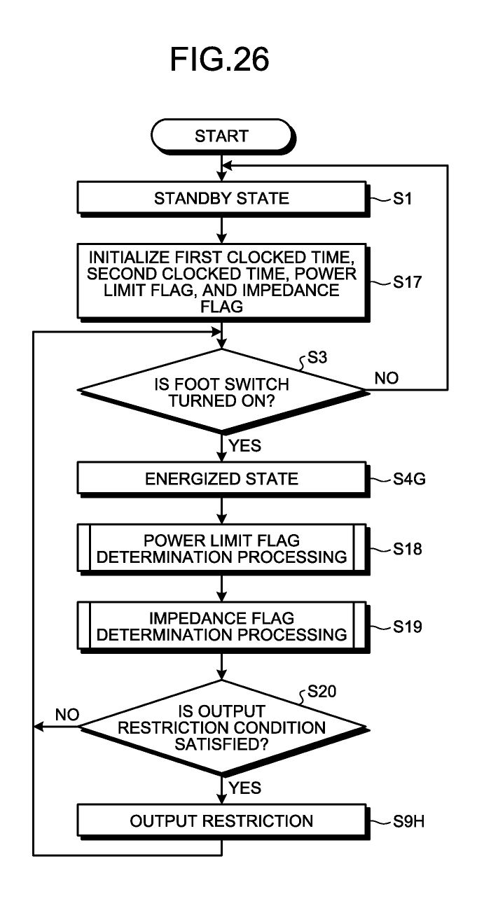

FIG. 26 is a flowchart illustrating operation of the control apparatus 3H.

After the power supply (not illustrated) of the thermal energy treatment apparatus 1H is turned on by the operator to set the energy treatment tool 2H in a standby state (step S1), the control apparatus 3H executes initialization of the first clocked time clocked by the power value determination unit 3321, the second clocked time clocked by the impedance value determination unit 3328, the power limit flag, and the impedance flag (step S17).

After step S17, the control apparatus 3H determines whether the foot switch 4 is turned on (step S3) similarly to the case of the above-described third embodiment, and executes switching to the energized state of the energy treatment tool 2H (step S4G).

After step S4G, the state determination unit 332H executes power limit flag determination processing (step S18) as described below.

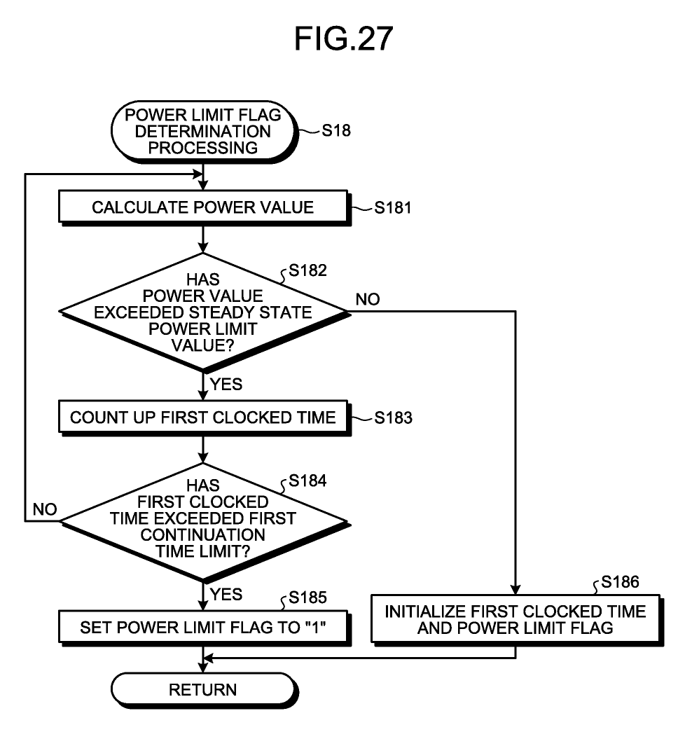

FIG. 27 is a flowchart illustrating the power limit flag determination processing (step S18).

First, the power value determination unit 3321 calculates a power value being supplied (alternating current energization) to the heating element 922 based on the current value and the voltage value detected by the sensor 32 (step S181).

After step S181, the power value determination unit 3321 executes determination (step S182) as to whether the power value has exceeded the steady state power limit value similarly to steps S6 and S7 described in the first embodiment. When it is determined that the power value has exceeded the steady state power limit value (step S182: Yes), the power value determination unit 3321 counts up the first clocked time (step S183).

After step S183, the first time determination unit 3322A determines (step S184) whether the first clocked time has exceeded the first continuation time limit similarly to step S8 described in the first embodiment.

When it is determined that the first clocked time has not exceeded the first continuation time limit (step S184: No), the control apparatus 3H returns to step S181.

In contrast, when it is determined that the first clocked time has exceeded the first continuation time limit (step S184: Yes), the first time determination unit 3322A sets the power limit flag to “1” (step S185). Thereafter, the control apparatus 3H returns to the main routine illustrated in FIG. 26.

When it is determined in step S182 that the power value has not exceeded the steady state power limit value (step S182: No), the control apparatus 3H initializes the first clocked time and the power limit flag (step S186). Thereafter, the control apparatus 3H returns to the main routine illustrated in FIG. 26.

After step S18, the state determination unit 332H executes impedance flag determination processing (step S19) as described below.

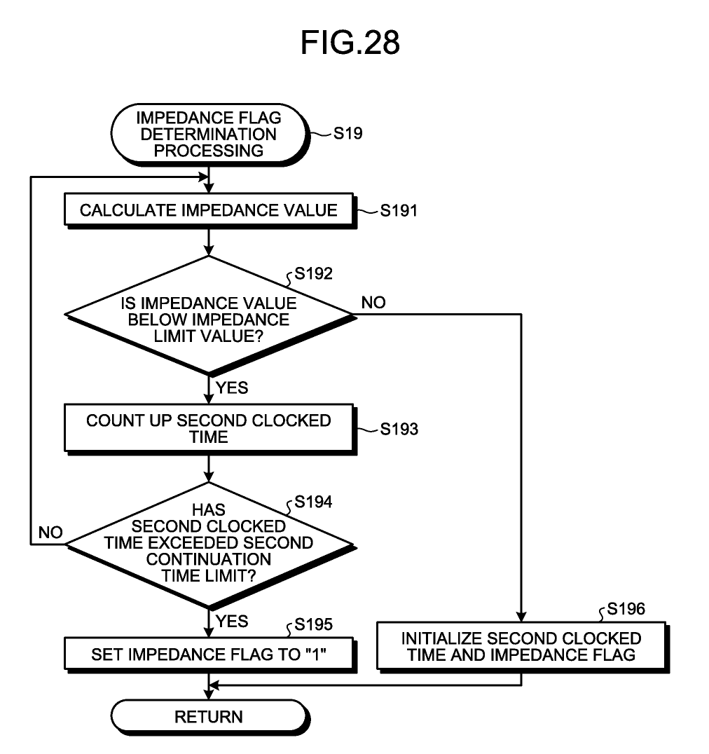

FIG. 28 is a flowchart illustrating the impedance flag determination processing (step S19).

First, the impedance value determination unit 3328 calculates an impedance value of the heating element 922 (step S191) similarly to steps S16 and S6G described in the third embodiment, and determines whether the impedance value is below the impedance limit value (Step S192).

When it is determined that the impedance value is below the impedance limit value (step S192: Yes), the control apparatus 3H counts up the second clocked time similarly to step S7 described in the third embodiment (step S193).

After step S193, the second time determination unit 3322B determines (step S194) whether the second clocked time has exceeds the second continuation time limit similarly to step S8 described in the third embodiment.

When it is determined that the second clocked time has not exceeded the second continuation time limit (step S194: No), the control apparatus 3H returns to step S191.

In contrast, when it is determined that the second clocked time has exceeded the second continuation time limit (step S194: Yes), the second time determination unit 3322B sets the impedance flag to “1” (step S195). Thereafter, the control apparatus 3H returns to the main routine illustrated in FIG. 26.

Moreover, when it is determined in step S192 that the impedance value has not exceeded the impedance limit value (step S192: No), the control apparatus 3H initializes the second clocked time and the impedance flag (step S196). Thereafter, the control apparatus 3H returns to the main routine illustrated in FIG. 26.

After step S19, the output restriction unit 333H reads the power limit flag and the impedance flag stored in the memory (not illustrated) in the control apparatus 3H and determines whether the output restriction condition (the power limit flag is “1” and the impedance flag is “0”) is satisfied (step S20).

Steps S18 to S20 correspond to the state determination step according to the present disclosure.

When it is determined that the output restriction condition is not satisfied (step S20: No), the control apparatus 3H returns to step S3.

In contrast, when it is determined that the output restriction condition is satisfied (step S20: Yes), the output restriction unit 333H controls operation of the thermal energy output unit 31 to restrict an output value (power value) to be supplied (alternating current energization) to the heating element 922 (output restriction) (step S9H: output restriction step).

Note that the output restriction of step S9H is executed until the foot switch 4 is turned off at step S3 (step S3: No) and switched to the standby state (step S1), similarly to the above-described first and third embodiments.

With the thermal energy treatment apparatus 1H according to Modification 3-1 described above, the following effects would be achieved in addition to the similar effects as those of the above-described third embodiment.

In the thermal energy treatment apparatus 1H according to modification 3-1, it is possible to determine whether the treatment is performed in an environment with a large heat capacity by executing step S18 and to determine whether the distal end of the energy treatment tool 2H is immersed in the liquid up to the pair of lead wire connecting units 9221 by executing step S19. That is, when the distal end of the energy treatment tool 2H is immersed in the liquid up to the pair of lead wire connecting units 9221, the heat of the pair of lead wire connecting units 9221 is dissipated to the liquid, leading to suppressing an occurrence of an overheated state of the pair of lead wire connecting units 9221. Therefore, output restriction is to be executed exclusively in cases where it is determined by executing step S18 that the treatment is performed in an environment with a large heat capacity, and where it is determined by executing step S19 that the distal end of the energy treatment tool 2H is not immersed in the liquid up to the pair of lead wire connecting units 9221, making it possible to avoid execution of unnecessary output restriction.

### Other Embodiments

Embodiments have been described hereinabove; however, the present disclosure is not intended to be limited to the above-described first to third embodiments and their modifications 1-1 to 1-3, 2-1, 2-2, and 3-1.

In the above-described first to third embodiments and modifications 1-1 to 1-3, 2-1, 2-2, and 3-1, the therapeutic energy applying structures 9, 9G, and 9H are provided on the holding member 8 alone. The present disclosure, however, is not limited thereto, and it is allowable to employ a configuration in which the therapeutic energy applying structures 9, 9G, and 9H are provided also on a holding member 8′.

In the first to third embodiments and the modifications 1-1 to 1-3, 2-1, 2-2, and 3-1, the therapeutic energy applying structures 9, 9G, and 9H are configured to apply thermal energy to living tissues. The present disclosure, however, is not limited thereto, and it is also allowable to configure to apply high-frequency energy or ultrasonic energy in addition to the thermal energy.

In the first embodiment and the modifications 1-1 to 1-3 described above, the waveforms (behavior) of the current value and the voltage value supplied (energized) to the heating element 922 when the treatment is performed in an environment with a large heat capacity are waveforms similar to the waveform of the power value. For this reason, it is allowable to also employ a current value or a voltage value other than the power value, as the index value according to the present disclosure

In the above-described second embodiment (FIG. 16), the output restriction (step S9) may be executed at a timing when the detected temperature exceeds the temperature limit value (step S6D: Yes). That is, steps S7 and S8 may be omitted.

In the above-described third embodiment (FIG. 23), the output restriction (step S9) may be executed at the timing when the impedance value is below the impedance limit value (step S6G: Yes). That is, steps S7 and S8 may be omitted.

Moreover, in modification 3-1 (FIG. 28), the impedance flag may be set to “1” (step S195) at the timing when the impedance value is below the impedance limit value (step S192: Yes). That is, steps S193 and S194 may be omitted.

In the above-described third embodiment and its modification 3-1, the heating unit 9222 is caused to generate heat by alternating current energization. The present disclosure, however, is not limited thereto, and the heating unit 9222 may be caused to generate heat by energization with direct current as in the first embodiment, or the like, and it is allowable to configure such that energization is switched to alternating current energization exclusively at the time of detecting the impedance value.

In the above-described first to third embodiments and the modifications 1-1, 1-2, 2-1, 2-2, and 3-1, it is allowable to configure so as to maintain a standby state without switching to the energized state when the output restriction is executed and thereafter the state has been switched to the standby state and when the timing at which the foot switch 4 is turned on is within a predetermined period after executing the output restriction.

According to the thermal energy treatment apparatus and the method of operating the thermal energy treatment apparatus according to the present disclosure, there is an effect of achieving prevention of an occurrence of an overheated state of the connecting unit.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A thermal energy treatment apparatus comprising:
an insulating substrate having a longitudinal axis;
a heating element provided on the insulating substrate, the heating element comprising
a heater configured to generate heat by energization, a resistance value of the heater having per unit length in a direction of the longitudinal axis being a first resistance value, and
a connector configured to be conducted to the heater, a resistance value of the connector per unit length in the direction of the longitudinal axis being a second resistance value smaller than the first resistance value; and
a controller configured to
determine a state of the connector based on an index value of a temperature of the connector, and
restrict an output value of energization of the heater based on the determined state of the connecting unit.

2. The thermal energy treatment apparatus according to claim 1, further comprising a heat transfer plate configured to transmit heat from the heater to a living tissue and having a length shorter than the insulating substrate in the longitudinal axis direction,
wherein the heater is arranged to face the heat transfer plate, and
the connector is provided on the insulating substrate protruding from the heat transfer plate in the longitudinal axis direction.

3. The thermal energy treatment apparatus according to claim 1,
wherein the controller determines a state of the connector using at least one of:
one of a current value, a voltage value, and a power value energized to the heater;
a temperature of the connector; and
an impedance value of the heater, as the index value.

4. The thermal energy treatment apparatus according to claim 1, further comprising a first detector configured to detect one of a current value, a voltage value, and a power value energized to the heater,
wherein the index value is one of the current value, the voltage value, and the power value detected by the first detector.

5. The thermal energy treatment apparatus according to claim 1, further comprising a temperature detector configured to detect a temperature of the connector, wherein the index value is the temperature detected by the temperature detector.

6. The thermal energy treatment apparatus according to claim 5,
wherein the controller compares the temperature detected by the temperature detector with a first threshold, and
the controller restricts the output value when the controller determines that the temperature has exceeded the first threshold.

7. The thermal energy treatment apparatus according to claim 1,
wherein the controller is configured to:
compare the index value with the first threshold and clock a time during which the index value continuously exceeds the first threshold;
compare the clocked time with a second threshold; and
restrict the output value when the controller determines that the time has exceeded the second threshold.

8. The thermal energy treatment apparatus according to claim 1,
wherein the controller compares an integrated value of the index value with a third threshold and restricts the output value when the controller determines that the integrated value has exceeded the third threshold.

9. The thermal energy treatment apparatus according to claim 1, further comprising a second detector configured to detect an impedance value of the heating element in a state where the heater is energized with alternating current,
wherein the heating element is configured to enable a portion of the heating element to come in contact with an external liquid, and
the index value is the impedance value detected by the second detector.

10. The thermal energy treatment apparatus according to claim 9,
wherein the controller compares the impedance value detected by the second detector with a fourth threshold and restricts the output value when the controller determines that the impedance value is below the fourth threshold.

11. The thermal energy treatment apparatus according to claim 9, further comprising a first detector configured to detect one of a current value, a voltage value, and a power value energized to the heater,
wherein the index value includes an impedance value detected by the second detector, and further includes one of the current value, the voltage value, and the power value detected by the first detector,
the heating element is configured such that the connector alone is capable of coming in contact with an external liquid,
the controller is configured to:
compare one of the current value, voltage value, and the power value detected by the first detector with the first threshold and clocks a time during which one of the current value, the voltage value, and the power value continuously exceeds the first threshold;
compare the clocked time with the second threshold; and
compare the impedance value detected by the second detector with the fourth threshold,
wherein the controller restricts the output value when the controller determines that the clocked time has exceeded the second threshold and determines that the impedance value is below the fourth threshold.

12. The thermal energy treatment apparatus according to claim 1,
wherein the controller restricts the output value by stopping energization to the heater.

13. The thermal energy treatment apparatus according to claim 1, further comprising an operation receiver configured to receive user operation to shift from a standby state in which energization to the heater is stopped to an energized state that energizes the heater,
wherein the controller is configured to shift from the standby state to the energized state when the operation receiver has received the user operation, and
when the operation receiver has received the user operation within a predetermined period after the output value is restricted by the controller, the controller maintains the standby state.

14. The thermal energy treatment apparatus according to claim 1, further comprising a notification unit configured to notify predetermined information,
wherein the controller is configured to activate the notification unit when the output value is restricted by the controller.

15. The thermal energy treatment apparatus according to claim 1, further comprising:
an output unit configured to apply a voltage to the heater under control of the controller; and
a first detector configured to detect one of a current value, a voltage value, and a power value energized from the output unit to the heater.

16. A method of operating a thermal energy treatment apparatus including: an insulating substrate; and a heating element provided on the insulating substrate, the heating element including a heater having a first resistance value and configured to generate heat by energization, and a connector having a second resistance value smaller than the first resistance value and configured to be conducted to the heater, the method comprising:
energizing the heater via the connector;
determining a state of the connector based on an index value of a temperature of the connector; and
restricting an output value of energization to the heater based on the determined state of the connector.
